(19) United States
(12) Patent Application Publication
(10) Pub. No.: US 2025/0017960 A1
(43) Pub. Date: Jan. 16, 2025

(54) COMPOSITION AND METHOD OF VALENCY CONTROLLED RECEPTOR SYSTEMS FOR CELL ENGINEERING AND THERAPY

(71) Applicant: The Board of Trustees of the Leland Stanford Junior University, Stanford, CA (US)

(72) Inventors: Lei S. Qi, Stanford, CA (US); Paul B. Finn, Stanford, CA (US)

(21) Appl. No.: 18/704,007

(22) PCT Filed: Nov. 15, 2022
(86) PCT No.: PCT/US2022/079889

§ 371 Date: Apr. 23, 2024

Related U.S. Application Data
(60) Provisional application No. 63/279,457, filed on Nov. 15, 2021.

(51) Int. Cl. A61K 35/17; A61K 39/00; A61P 35/00; C07K 14/705; C07K 14/725; C07K 16/28

(52) U.S. Cl. CPC A61K 35/17; A61K 39/4611; A61K 39/4631; A61K 39/464412; A61P 35/00; C07K 14/7051; C07K 14/70521; C07K 16/2803; A61K 2239/13; A61K 2239/17; A61K 2239/21; C07K 2317/622; C07K 2319/03

(57) ABSTRACT

Provided herein are valency controllable receptor polypeptides configured to oligomerize upon recognition of a custom input. The receptors include an extramembrane signal recognition domain configured to recognize an extramembrane signal different from the custom input, a valency control module configured to recognize the custom input and induce oligomerization of the polypeptide, and an intramembrane signaling domain configured to modulate one or more intramembrane pathways. The provided receptors are particularly useful for engineered cell therapies. Also provided are systems and host cells including the disclosed receptors, and methods for using the disclosed materials.

### CROSS-REFERENCE TO RELATED APPLICATIONS

The present application claims priority to U.S. Provisional Application No. 63/279,457 filed Nov. 15, 2021, the full disclosure of which is incorporated by reference in its entirety for all purposes.

### BACKGROUND

Cell therapy is revolutionizing the ways in which diseases can be treated. By utilizing cells as a therapy, the natural complex functions of the human body can be reprogrammed to target sources and symptoms of diseases, thereby opening an enormous space of possible therapeutic modalities that were previously unavailable using old paradigms. For example, in the adoptive cell therapy of cancer treatment, immune cells are extracted from the patient, engineered with new functionality through molecular addition or genetic modification, and reintroduced to the patient to eliminate cancer cells. A successful clinical example of this approach is CAR T cell therapy, a strategy for treating liquid cancers in patients who have relapsed or become refractory to traditional forms of therapy such as radiation and chemotherapy. In this type of therapy, T cells are engineered with a chimeric antigen receptor (CAR) that imbues the T cells with the ability to target a cancer and subsequently activate native T cell functionality to target and kill the malignancy. The success of these engineered cell therapies relies on rewiring natural immune activation pathways to different disease relevant targets through genetic manipulation. The engineered cells can survey the entire system of a patient and mount immune responses against cancer cells, resulting in remission rates that were previously unobtainable with other forms of therapy. However, many challenges and unfilled niches exist within this important therapeutic space.

First, effective CAR T cell response requires high target antigen density and often exhibits tumor escape. Antigen density currently must be above a certain threshold for therapies targeting these antigens to be effective. Cancers often evolve over the course of CAR T treatment, however, to reduce antigen density and escape recognition by the cell therapy. Accordingly, while initial responses to current approved cell therapies can be high, most patients relapse with post-treatment disease progression associated with diminished antigen density. Low antigen density on target cells therefore makes current CAR therapies ineffective against FDA-approved blood-based indications. Such drawbacks will similarly affect treatments targeting the variable and heterogeneous environment of solid tumors, the dynamic antigens of viral infections, or the less differentiating antigens of autoimmunity and ageing.

Second, engineered immune cell activity and activation is currently only controlled by tumor surface expressed disease signals. These therapies lack the ability to recognize otherwise equally relevant soluble factors such as disease-associated soluble antigens or pharmacological interventions. As one result of this limitation, the engineered cells act autonomously and cannot be controlled from patient to patient by soluble factors administered by a physician or through the presence of soluble antigen. The lack of external control by a physician can lead to cytokine release syndrome or rapid T cell exhaustion associated with poor efficacy. The lack of control through soluble antigens can lead to on-target off-tumor targeting due to the small space of disease differentiating surface antigens or low specificity of T cell spatial localization. Such undesired off-tumor targeting has been shown in some cases to cause organ-level toxicities. Concepts for designing clinically proven small molecules to modulate and tune the activity of CAR T cells, mitigate T cell exhaustion, and enhance disease localization are thus urgently needed.

Third, there has been limited success incorporating additional signaling domains into CAR. For example, current CAR designs are based on signaling in an in cis format, while T cells utilize in trans receptor combinations to activate, inhibit and/or regulate T cell function. Non-natural signaling formats can significantly alter signaling potential, and can limit the pool of inhibitory and co-stimulatory domains that can be utilized and discovered. The vast majority of synthetic tools activate only one specific receptor signaling pathway in T cells with limited efficacy, whereas multiple signaling pathways in a natural T cell give rise to differential signaling dynamics and therefore different immune responses. An ability to define other molecular compositions, for example those with dual receptors, could therefore be a key innovative aspect providing new features to immune and bringing cell therapy to unmet indications.

In view of these observations and results, there is a need in the art for new systems for engineering cells that are useful in improved therapies.

### BRIEF SUMMARY

The disclosure herein provides a set of solutions to address challenges associated with engineered cell therapies that otherwise require high target antigen density and cannot be controlled by soluble factors. These solutions include molecular designs and compositions according to particular engineered receptor designs, and methodologies of using such receptors and systems including these receptors for improved cell therapies. The provided materials and methods have broad applicability with both adaptive and innate immune cells, and enable clinical features that can impact broad disease indications.

In one aspect, the disclosure provides a valency controllable receptor polypeptide. The valency controllable receptor polypeptide includes an extramembrane signal recognition domain having an ability to recognize an extramembrane signal. The valency controllable receptor polypeptide further includes a valency controller module. The valency controller module includes a controller domain connected to the extramembrane signal recognition domain. The controller domain is configured to recognize a custom input different from the extramembrane signal. The valency controller module further includes a transmembrane domain connected to the controller domain. The valency controller module is configured to oligomerize the valency controllable receptor polypeptide upon recognition of the custom input by the controller domain. The valency controllable receptor polypeptide further includes one or more intramembrane signaling domains connected to the transmembrane domain.

In another aspect, the disclosure provides a valency controllable receptor system. The valency controllable receptor system includes two or more valency controllable receptor polypeptides. Each of the two or more valency controllable receptor polypeptides is independently a valency controllable receptor polypeptide as disclosed herein.

In another aspect, the disclosure provides another valency controllable receptor system. The valency controllable receptor system includes a first valency controllable receptor polypeptide. The first valency controllable receptor polypeptide is a valency controllable receptor polypeptide as disclosed herein. The valency controllable receptor system further includes a second valency controllable receptor polypeptide. The second valency controllable receptor polypeptide includes a second valency controller module. The second valency controller module includes a second controller domain configured to recognize a second custom input different from the extramembrane signal. The second valency controller module further includes a second transmembrane domain connected to the second controller domain. The second valency controller module is configured to oligomerize the second valency controllable receptor polypeptide upon recognition of the second custom input by the second controller domain. The second valency controllable receptor polypeptide further includes one or more intramembrane signaling domains connected to the second transmembrane domain.

In another aspect, the disclosure provides a host cell. The host cell includes a valency controllable receptor as disclosed herein, or a valency controllable receptor system as disclosed herein.

In another aspect, the disclosure provides a method for modulating an intramembrane pathway. The method includes providing a valency controllable receptor polypeptide as disclosed herein, or a valency controllable receptor system as disclosed herein.

In another aspect, the disclosure provides a method for preventing or treating a disease in a subject. The method includes administering to the subject an amount of a valency controllable receptor polypeptide as disclosed herein, a valency controllable receptor system as disclosed herein, or a host cell as disclosed herein.

In another aspect, the disclosure provides a method for healing a wound in a subject. The method includes administering to the subject an amount of the valency controllable receptor polypeptide as disclosed herein, a valency controllable receptor system as disclosed herein, or a host cell as disclosed herein.

### BRIEF DESCRIPTION OF THE DRAWINGS

The present disclosure is described in detail below with reference to the appended drawings

FIG. 1 is a schematic illustration of a general design for incorporating a valency controller module into an existing synthetic receptor polypeptide to create a valency controllable receptor polypeptide in accordance with an embodiment.

FIG. 2 is a schematic illustration of a valency controllable receptor system in accordance with an embodiment. The valency controllable receptor system includes multiple copies of the same valency controllable receptor polypeptide, where the multiple copies oligomerize upon contact with an extracellular valency control ligand, thereby modulating the activity of one or more intramembrane pathways.

FIG. 3 is a schematic illustration of another valency controllable system in accordance with an embodiment. The valency controllable receptor system includes multiple different valency controllable receptor polypeptides, where the multiple different polypeptides oligomerize upon contact with an extracellular valency control ligand, thereby modulating the activity of one or more intramembrane pathways.

FIG. 4 presents a schematic illustration of a valency controllable receptor polypeptide based on the 1928z CAR receptor (top) and a graph showing enhanced signaling of the valency controllable receptor polypeptide in Jurkat T cells (bottom). The data of the graph was measured by culturing the engineered Jurkat T cells with or without CD19+ Nalm6 cells and with or without valency control ligand (VCL) treatment. NT indicates no treatment.

FIG. 5 is a graph showing the performance of the valency controllable receptor polypeptide of FIG. 4 in human primary T cells in co-culture with CD19+ (low) Nalm6 cells, with or without VCL treatment. NT is no treatment.

FIG. 6 presents a schematic illustration of a valency controllable receptor polypeptide based on the 19BBz CAR receptor (top) and a graph showing enhanced signaling of the valency controllable receptor polypeptide in Jurkat T cells (bottom). The data of the graph was measured by culturing the engineered Jurkat T cells with or without CD19+ Nalm6 cells and with or without VCL treatment. NT indicates no treatment.

FIG. 7 is a graph showing the performance of the valency controllable receptor polypeptide of FIG. 6 in human primary T cells in co-culture with CD19+ (low) Nalm6 cells, with or without VCL treatment. NT is no treatment.

FIG. 8 is a schematic illustration of a valency controller module configuration useful for valency controllable receptor polypeptides having intramembrane valency controller domains capable of recognizing a intramembrane custom input in accordance with an embodiment.

FIG. 9 is a schematic illustration of a valency controller module configuration useful for valency controllable receptor polypeptides having extramembrane valency controller domains capable of recognizing an extramembrane custom input in accordance with an embodiment.

FIG. 10 is a schematic illustration of SNAP valency controllable receptor (VCR) containing an extracellular SNAP-tag protein, a CD28 transmembrane domain (CD28tm), a CD28 cytoplasmic (CD28cy) domain, and CD3, cytoplasmic domain. SNAP-VCRs are conjugated to single stranded DNA (ssDNA) using a benzyl guanidine (BG) modified oligo.

FIG. 11 is an illustration showing utilization of DNA origami to create a trivalent ssDNA structure (ssDNA Y-DNA) that induces clustering of the SNAP-VCR of FIG. 10.

FIG. 12 is a graph of data showing that VCR-mediated T cell activation is induced in Jurkat cells only when both the receptor of FIG. 10 has been conjugated to ssDNA and the ssDNA Y-DNA of FIG. 11 is present.

FIG. 13 an illustration showing use of DNA origami to generate a trivalent biotin structure (biotin Y-DNA) that oligomerizes a VCR with an extracellular monomeric streptavidin (mSA).

FIG. 14 is a graph showing that mSA VCR Jurkats exhibit biotin Y-DNA dose dependent activation.

FIG. 15 is a graph showing that mSA VCRs in Jurkat reporter cell lines induce NFAT signaling only in the presence of biotin Y-DNA.

FIG. 16 is a graph showing that mSA VCRs in Jurkat reporter cell lines induce NFκB signaling only in the presence of biotin Y-DNA.

FIG. 17 presents the structures of biotin, JQ1, and FITC end groups of different trivalent Y-DNA, and a graph showing results of using the Y-DNA to induce T cell activation in Jurkats for VCRs containing extracellular mSA (with biotin Y-DNA), BRD4 (with JQ1 Y-DNA), FluA anticalin (with FITC Y-DNA), and a FITC scFv (with FITC Y-DNA).

FIG. 18 presents an illustrations showing that DNA origami can be programmed to create Y-DNA with different biotin valencies, and a graph showing probe the effects of different valencies on VCR-mediated T cell activation.

FIG. 19 is a graph showing that shorter distances between the biotins of a Y-DNA structure can increase T cell activation.

FIG. 20 is a graph showing that programming DNA origami to alter the distance between biotins and create a closer proximity can induce higher IL-2 secretion upon T cell activation in Jurkats.

FIG. 21 is an illustration of DNA origami programmed to create I-DNA (bivalent), Y-DNA (trivalent), or X-DNA (tetravalent) scaffolds for biotin.

FIG. 22 is a graph showing that CD69 expression in Jurkat cells expressing mSA-VCR and dosed with DNA were comparable between X- and Y-DNA but were reduced using I-DNA.

FIG. 23 is a graph showing that NFAT signaling in Jurkat cells expressing mSA-VCR and dosed with DNA were comparable between X- and Y-DNA but were reduced using I-DNA.

FIG. 24 is a graph showing that the pattern of observations in FIGS. 22 and 23 translated to primary human T cells where I-DNA no longer induced T cell activation but X- and Y-DNA did. Data from two donors.

FIG. 25 shows a scheme for synthesizing a tetravalent biotin compound using a generation 0 PAMAM dendrimer scaffold to create a small molecule version of the biotin X-DNA (4° Bio).

FIG. 26 is a graph showing that the 4° Bio of FIG. 25 induced NFAT signaling in VCR engineered Jurkat reporter cell lines.

FIG. 27 is a graph showing that VCRs containing a CD28 costimulatory domain and CD3ζ signaling domain (28ζ) and dosed with 4° Bio favored NFAT signaling.

FIG. 28 is a graph showing that VCRs containing a 4-1BB costimulatory domain and CD3ζ signaling domain (28ζ) and dosed with 4° Bio favored NFκB.

FIG. 29 is a graph showing that VCRs containing only a 4-1BB (BB) intracellular domain could induce NFκB signaling and synergize with antibody mediated TCR signaling.

FIG. 30 is an illustration of a monomeric version of VCR created by mutating the hinge and transmembrane domains. Monomeric VCRs create receptor clusters that are dictated by the valency of the drug. The original dimeric VCRs can create much larger clusters through drug mediated daisy chaining of many clusters.

FIG. 31 is an illustration of mutations in the hinge and transmembrane made to design a monomeric mSA-VCR.

FIG. 32 is a graph showing that when using the tetravalent biotin (4° Bio), the monomeric VCR performed slightly worse than dimeric VCR.

FIG. 33 is a graph showing that when using a bivalent drug, the monomeric VCR can fail to signal, indicating that the drug can induce VCR superclusters via receptor daisy chaining.

FIG. 34 is an illustration of VCR chimeric antigen receptors (VCR-CARs), which are VCRs fused with an scFv to enable mechanotransduction-mediated T cell activation. Dosing these VCR-CARs with a multivalent drug induces clustering to enhance T cell signaling upon antigen binding.

FIG. 35 is a graph showing a comparison of NFAT signaling with VCRs fused to a CD19 targeting scFv (VCR-19BBz and VCR-1928z) with and without 4° Bio in the absence of antigen.

FIG. 36 is a graph showing that VCRs fused to a CD19 targeting scFv (VCR-19BBz) only activate NFAT signaling in Jurkat reporter cell lines in the presence of CD19+ Nalm6 cells, and are enhanced by dosing with 4° Bio.

FIG. 37 is a graph showing that HER2 targeted VCR-CAR T cells (VCR-HER2BBz) are potent in the presence of HER+ Nalm6 cells and enhanced by treatment with 4° Bio.

FIG. 38 is a graph plotting results of a live cell microscopy based killing assays, showing that drug dosed VCR-19BBz engineered primary human T cells are more cytotoxic to CD19+ Nalm6 cells when treated with 4° Bio (1:1 effector to target, E:T, ratio).

FIG. 39 is a graph showing that final target cell densities in the assays of FIG. 38 were dramatically reduced with VCR-19BBz engineered primary T cells across multiple donors.

FIG. 40 is a graph showing improved cytotoxicity of VCR-HERBBz engineered primary T cells against HER2+ 143b spheroids.

FIG. 41 presents the structures of a series of tetravalent biotin drugs designed and synthesized with different linkers and conjugated to a generation 0 PAMAM dendrimer to improve bioavailability.

FIG. 42 is a graph plotting the bioavailability half-life of the compounds of FIG. 41.

FIG. 43 is a graph plotting the percent of cells activating the NFAT pathway at various concentrations of compounds of FIG. 41.

FIG. 44 is a graph showing that PBF004 of FIG. 41 exhibited potent activation of VCR-19BBz Jurkat reporter cell lines compared to PBF001 and PBF002.

FIG. 45 is a graph showing that PBF004 remains in the optimal T cell activation window for 8-24 hours in mouse serum.

FIG. 46 is an illustration of a protocol in which 1×10⁶ Nalm6 cells expressing luciferase were i.v. injected into mice and, 4 days later. T cells (mock) or VCR-19BBz engineered T cells (3×10⁶ receptor positive) were i.v. injected. Some mice were dosed by i.p. injection with PBF004 daily for 21 days and tumor burden was measured by bioluminescence imaging (BLI) thrice weekly.

FIG. 47 is a graph showing that dosed mice carrying VCR-CAR T cells according to the protocol of FIG. 46 controlled tumor burden better than both mice treated with mock and mice treated with only VCR-CAR T cells (no drug dosing).

FIG. 48 is a graph showing that the results described in FIG. 47 led to enhanced survival for mice with CD19-high Nalm6 cancer cells and dosed VCR-CAR T cells and dosed with drug mediated inducible control.

FIG. 49 is a graph plotting observations from live cell microscopy assays demonstrating that dosing VCR-CAR T cells targeted to a CD19-low or high expressing Nalm6 cell line rescues its cytotoxicity.

FIG. 50 is a graph showing that drug activated VCR-CART cells controlled CD19-low Nalm6 tumor burden better than mice treated with mock and mice treated with only VCR T cells (no drug dosing).

FIG. 51 is a graph showing that only drug-activated VCR-CAR T mice survived the CD19-low Nalm6 study.

### DETAILED DESCRIPTION OF THE INVENTION

The inventors have developed a technology to convert existing or newly discovered or developed natural or synthetic transmembrane receptors into valency controllable receptors. In some embodiments, these valency controllable receptors maintain the ability to recognize the cognate input that the original natural or synthetic transmembrane receptor was capable of binding to, but also include an additional valency controller module giving the valency controllable receptors an ability to additionally bind to a different custom input. Upon binding to this custom input, the valency controller module causes the valency controllable receptor to oligomerize. This oligomerization event increases a localized concentration of the valency controllable receptors in the membrane, advantageously improving the capability of the receptors to recognize the cognate input, even if present in a low amount. The concentrating of the valency controllable receptors through oligomerization also advantageously improves properties for transmitting an extramembrane signal input across a membrane, e.g., a cell or organelle membrane, to activate intramembrane signal pathways. Because the oligomerization is triggered by a custom input that is different from the cognate input of the original natural or synthetic transmembrane receptor, the valency controllable receptor polypeptide can be designed or configured in a way that does not disturb the original receptor binding characteristics. The valency controllable receptor polypeptide can also be designed or configured to have an oligomerization response that is tunable in response to the presence and concentration of the custom input used to contact the valency controller module.

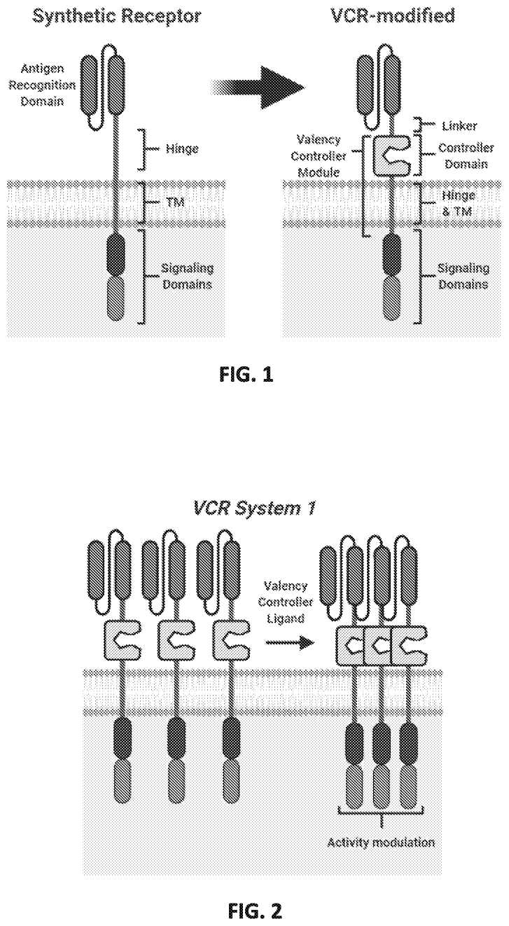

As shown in FIG. 1, the valency controller module of the valency controllable receptor polypeptides disclosed herein can include one or more controller domains that recognize a custom input. The valency controller module can also include a hinge/transmembrane domain containing zero, one, or more hinge regions fused to the N or C terminus of the controller domain. The valency controller domain can also include one or more linker units for fusing the valency controller module to an extramembrane signal recognition domain, e.g., the natural or synthetic transmembrane receptor. The valency controller module can induce oligomerization of the modified natural or synthetic receptor upon treatment with the custom input, which clusters multiple valency controllable receptor polypeptides of a valency controllable receptor system together to activate, inhibit, modulate and/or enhance downstream signals for, e.g., signal pathway control, genome control, or cell function control.

A valency controllable receptor system can include, as show in FIG. 2, a single type of valency controllable receptor polypeptide that is a modified form of a natural or synthetic receptor and that retains the ability to recognize the cognate input or ligand. A valency controllable receptor system can alternatively include, as shown in FIG. 3, two or more types of valency controllable receptor polypeptides, at least one of which is a modified form of a natural or synthetic receptors retaining the ability to recognize the cognate input or ligand. In each system, the valency controller module of the valency controllable receptor polypeptide retaining the ability to recognize the cognate input or ligand synergizes with the cognate receptor signaling mechanism, e.g., mechanical force, such that input-dependent induced oligomerization forms a complex of valency controllable receptor polypeptides.

Among the many other benefits provided by the valency controllable receptor polypeptide of the systems and methods disclosed herein is that the receptor can be activated in an inducible fashion by the presence or absence of certain signals that can be either endogenous or exogenous. This induction can be reversed by removal of the signal, addition of a second signal, or addition of a competing signal, to restore the receptor status or a host cell status to its original state. Also, depending on the concentration or intensity of the signal, the receptor system can be controlled precisely by the amount of added signals to fine-tune its activity.

The valency controllable receptor polypeptide further allows the repurposing of many available small molecule drugs, peptides, nanobodies, scFvs, or antibodies as safe and effective inducers. This includes those drugs that are approved by FDA or those that failed merely due to compromised bioavailability and efficacy. The receptor system does not require that the signal enters host cells, so small molecules or antibodies that cannot enter cells can also be used as inducers. This greatly expands the compatibility of diverse safe molecules with the provided methods. Moreover, multiple receptors with various inputs and outputs can be combined with each other or combined with other immune cell receptors, e.g., T cell- or B cell-specific receptors, to generate combinatorial functions. Importantly, when and where such receptor engineered cells are activated can be controlled by the location and administration of the extramembrane signal.

The valency controllable receptors and systems of the provided materials and methods thus provide several advantages and improvements over synthetic receptors and systems that were previously available. For example, unlike first-generation CAR T cell therapeutic receptors which consist of an extracellular cancer binding domain and an intracellular T cell activating domain, and which imbue weak stimulation to T cells and display poor efficacy in killing cancer, the receptors disclosed herein can use a custom input signal to better control a CAR T cell response upon target engagement through the activation of synergistic signaling pathways to aid in tumor clearance and disease elimination.

In some embodiments, the valency control ligand induces phase condensation of the valency controllable receptor polypeptides or valency controllable receptor polypeptide-fused molecules on the surfaces of host cells. The valency control ligand can be synthetic valency control molecule that can still trigger or enhance condensation of valency controllable receptor polypeptide molecules.

Second-generation CAR T cell therapeutic receptors include both a costimulatory domain and a T cell activation domain on their intracellular side. The costimulatory domain improves the therapeutic response of T cells to some extent by increasing T cell proliferation or cytotoxicity. However, there is no control of the therapeutic window of these engineered cells, and significant side-effects can be created in patients. Because the costimulatory domain is tied to the CAR, however, these second-generation cells have a binary “all or nothing” response resulting only in full activity when binding to a cancer. In contrast, the provided valency controllable receptors can afford control over the therapeutic window of an engineered cell, e.g., an engineered T cell. Moreover, the use of valency control with the provided systems can enhance activity against tumor targets having low antigen density. These improvements can better allow a healthcare professional to both maintain efficacy and increase patient safety during a more tunable treatment for tumor clearance and disease elimination.

T cell receptor (TCR)-engineered T cells can provide a cancer patient with a TCR that binds a particular marker indicative of a disease such as a specific type of cancer. TCR-engineered T cells genetically introduce the specific TCR into a patient's T cells, which are then reinfused into the patient to search for and destroy the targeted cancer. However, as with first-generation CARs, the cancer-binding signal insufficiently induces activation of the T cells. The provided valency controllable receptor polypeptide or system can be introduced to the patient's T cells along with the TCR to provide an important secondary signal that can allow the TCR therapy to work more effectively, and in a more tunable fashion.

In START CAR T cells, the extracellular binding domain and the intracellular costimulatory and activation domain are naturally separate, but a physician-given small molecule allows these separate pieces to dimerize and the T cell to perform its reprogrammed function. In the STOP CAR case, the extracellular and intracellular pieces are naturally dimerized, but a physician-given small molecule decouples the pieces to turn the CAR T cell off. In both cases, the small molecule drug must be cell permeable and the T cells have an “all or nothing” response. In the provided materials and methods, however, the custom input activator can be cell impermeable as the receptor can be engineered to be at the cell surface, significantly increasing the variety of molecules that can be used as an extramembrane signal. Further, the STOP or START CAR T cell systems are not capable of signal enhancement, and cannot overcome problems associated with low antigen density targeting. In contrast, the provided receptors and systems provide a solution to low antigen density problems, and can enhance signaling through not only receptor dimerization, but also higher order oligomerization. In addition, the provided receptors, when used in conjunction with a CAR, can titrate the CAR response. In doing so, cells engineered with the provided receptors and systems can always have some level of activity, ensuring that cancer fighting capabilities are always present and that a therapy is not cleared from the patient system due to lack of stimulation.

Another engineered receptor, iMC, can be used in conjunction with a CAR and can be dimerized with a small molecule to act as the secondary signal for CAR T cell killing of cancer. The residence of the small molecule binding domain of iMC in the cytoplasm, though, requires that its small molecule inducer be cell permeable, greatly reducing the space of drugs that can activate it. Also, the iMC receptor was designed to function with a specific, and clinically unproven, costimulatory domain. Beneficially, the provided valency controllable receptors are adaptable to many different costimulatory domains, including ones with FDA clearance and ones yet to be discovered. The provided receptors can also advantageously respond to activators that contain two or more interaction moieties, whereas iMC recognizes only a single rapalog activator moiety.

The synNotch receptor contains an extracellular cell surface binding domain and an intracellular orthogonal transcription factor, and is agnostic to cell-type and output. The receptor must, however, recognize surface-bound antigens to function. The provided valency controllable receptors can advantageously also recognize, for example, soluble factors created by the patient's body or administered as a drug. Furthermore, while the synNotch receptor output can be designed by genetically introducing a cellular program, the provided receptor can be connected to a multitude of natural pathways that activate various complex immune phenotypes. For example, the provided receptors can not only recognize both surface-bound antigens and soluble factors, but can also be configured to act according to an AND/OR gated format.

The first of two parts of the MESA engineered receptor includes an extracellular binding domain connected to a transmembrane domain and an intracellular protease. The second part of MESA includes the same extracellular binding domain connected to a transmembrane domain and an intracellular activation domain connected to the protease cleavage site. In this way, a signal forces oligomerization of the two parts, bringing the protease and the protease cleavage site in proximity, and inducing cutting of the activation domain from the receptor. The thus freed activation domain is then able to move about the cell and activate pathways. The activation domains of MESA are therefore limited to a small subset of transcription factors and other activators that can be controlled by cellular localization (such as nuclear exclusion). Unlike the provided valency controllable receptor, MESA cannot be used to activate the multitude of cell signaling responses naturally induced by oligomerization. In addition, the MESA receptor is a heterodimer requiring two genetic components for expression whereas the provided receptor requires only one polypeptide.

### Valency Controllable Receptor Polypeptides

In one aspect, a valency controllable receptor polypeptide is disclosed. The valency controllable receptor polypeptide includes at least three connected domains or modules—an extramembrane signal domain, a valency controller module, and an intramembrane signaling domain—that each perform a separate, but related, function. The extramembrane signal recognition domain of the valency controllable receptor polypeptide has an ability to recognize an extramembrane signal. The intramembrane signaling domain functions to transmit intramembrane signals in response to this recognition of the extramembrane signal by the extramembrane signal recognition domain. The valency controllable module has an ability to recognize a custom input that is distinct from the extramembrane signal, such that recognition of the custom input by the valency controllable module can lead to oligomerization of the valency controllable receptor polypeptide. This oligomerization clusters valency controllable receptor polypeptide, thereby enhancing the effectiveness of the extramembrane signal recognition and/or the intramembrane signal transmission.

The extramembrane signal domain, valency controller module, and intramembrane signaling domain of the provided valency controllable receptor polypeptide are connected to one another. As used herein, the term “connected” when used in reference to polypeptide domains, modules, regions, and the like indicates that these features are elements of the same polypeptide. Recited domains, modules, or other polypeptide elements can be connected directly adjacent to one another, such that the N or C terminus of one recited domain, module, or other element can be fused, e.g., covalently joined, to the C or N terminus of another recited domain, module, or element. Recited domains, modules, or other polypeptide elements can be connected indirectly to one another, such that the recited domains, modules, or other polypeptide elements can be separated by intervening sequences of the polypeptide, including those of one or more additional domains, modules, or other polypeptide elements.

In some embodiments, the oligomerization of the provided valency controllable receptor polypeptide upon recognition of the custom input by the controller domain of the valency controller module causes the valency controllable receptor polypeptide to oligomerize and form a dimer, e.g., a homodimer or heterodimer, In some embodiments, the valency controllable receptor polypeptide is configured to form a trimer. e.g., a homotrimer or heterotrimer, upon recognition of the custom input by the controller domain. In some embodiments, the valency controllable receptor polypeptide is configured to form a tetramer, e.g., a homotetramer or heterotetramer, upon recognition of the custom input. In some embodiments, the valency controllable receptor polypeptide is configured to form a higher-order oligomer upon recognition of the custom input by the controller domain. For example, a custom input based on a starburst dendrimer scaffold can be recognized by a controller domain to induce formation of a single oligomer complex with 16 valency controllable receptor polypeptides.

A wide variety of custom inputs can be recognized by a controller domain of a valency controllable receptor polypeptide as disclosed herein. In some embodiments, an extramembrane custom input is recognized by the controller domain, where the valency controller module is located in the extramembrane portion of the valency controllable receptor polypeptide. In some embodiments, an intramembrane custom input is recognized by the controller domain, where the valency controller module is located in the intramembrane portion of the valency controllable receptor polypeptide.

The provided valency controllable receptor polypeptide can be configured to recognize one or more of a wide variety of custom inputs inducing receptor oligomerization. In some embodiments, the custom input includes a valency control ligand capable of binding to the controller domain of the valency controllable receptor polypeptide. In some embodiments, the custom input includes a ligand that is a small molecule, e.g., a small molecule drug or a small molecule hormone. In some embodiments, the custom input consists of a small molecule. As used herein, the term “small molecule” refers to a chemical entity having a molecular weight less than 3,000 Daltons. The controller domain of the valency controllable receptor polypeptide can be configured, designed, or selected to recognize, for example, rimiducid (AP1903), darunavir, tamoxifen, estradiol, AP20187, or a symmetrical steroid. The small molecule valency control ligand recognized by the controller domain can be, for example, a monomer (e.g., caffeine or AP21967), a dimer (e.g., AP20187 or a synthetic Mcl-1 molecular glue), or a tetramer (e.g., a synthetic biotin dendrimer).

In some embodiments, the custom input includes a valency control ligand that is a metabolite. In some embodiments, the custom input consists of a metabolite. As used herein, the term “metabolite” refers to a chemical entity produced by one or more enzymatic or non-enzymatic reactions as a result of exposure of an organism to a chemical substance. The controller domain of the valency controllable receptor polypeptide can be configured, designed, or selected to recognize, for example, a cytokine, a chemokine, or a cancer-associated antigen. The controller domain can be configured to recognize a tumor marker derived from mucin-1 such as Carcinoma Antigen (CA) 15-3. The controller domain can be configured to recognize a tumor marker derived from mucin-16 such as CA 125. The controller domain can be configured to recognize amino acid metabolites such as Kynurenine.

In some embodiments, the custom input includes a valency control ligand that is an oligonucleotide. In some embodiments, custom input consists of an oligonucleotide. As used herein, the term “oligonucleotide” refers to a short nucleic acid molecule comprised of at least six covalently linked natural or chemically modified nucleosides. The controller domain of the valency controllable receptor polypeptide can be configured, designed, or selected to recognize an oligonucleotide comprising any combination of DNA nucleotides, RNA nucleotides, locked nucleic acid (LNA) nucleotides, or other synthetic nucleotide derivatives. In some embodiments, the custom input includes or consists of a ligand that is a longer polynucleotide. The oligonucleotide of the custom input can include, for example, DNA origami creating Y-DNA, X-DNA, and/or I-DNA. The oligonucleotide of the custom input can have a multimeric form, such as in a DNA origami structure. The oligonucleotide can have a hybrid form in which it is conjugated to a small molecule, such as biotin.

In some embodiments, the custom input includes a valency control ligand that is a peptide or protein. In some embodiments, the custom input consists of a ligand that is a peptide or protein. As used herein, the terms “peptide” and “protein” refer to polymers comprised of covalently linked natural or chemically modified amino acid residues. The controller domain of the valency controllable receptor polypeptide can be configured, designed, or selected to recognize, for example, cyclic peptides or linear peptides. The peptide of the custom input can be a naturally occurring protein, such as EGF. The peptide of the custom input can be a synthetic non-naturally occurring protein.

In some embodiments, the custom input includes a valency control ligand that is a polysaccharide. In some embodiments, the custom input consists of a ligand that is a polysaccharide. As used herein, the term “polysaccharide” refers to a polymer comprised of covalently linked natural or chemically modified sugar molecules. Polysaccharides include, for example, cellulose, hemicellulose, lignocellulose, starch, and the like.

In some embodiments, the custom input includes a valency control ligand that is another form of polymer. In some embodiments, the custom input includes or consists of a ligand that is a natural polymer. In some embodiments, the custom input consists of or comprises a ligand that is a synthetic polymer. In some embodiments, the custom input includes or consists of a ligand that is a dendrimer, i.e., a highly ordered, branched polymer. In some embodiments, the custom input includes or consists of a ligand that is a conjugate in which one or more, e.g., two or more, interaction molecules or moieties, e.g., small molecules, peptides, oligonucleotides, and/or polysaccharides, are conjugated to a polymer or dendrimer scaffold. In some embodiments, the custom input includes or consists of a ligand that is a conjugate in which biotin is conjugated to a polymer or dendrimer scaffold.

In some embodiments, the custom input includes a valency control ligand that is a lipid. In some embodiments, the custom input consists of a ligand that is a lipid. As used herein, the term “lipid” refers to a chemical entity having a hydrophilic moiety covalently attached to one or more hydrophobic moieties. Lipid molecules include, for example, fats, waxes, steroids, cholesterol, fat-soluble vitamins. monoglycerides, diglycerides, phospholipids, sphingolipids, glycolipids, cationic or anionic lipids, derivatized lipids, and the like. The controller domain of the valency controllable receptor polypeptide can be configured, designed, or selected to recognize modified lipids, e.g., lipids modified as a result of a cancer.

In some embodiments, the custom input includes a valency control ligand that is an antibody. In some embodiments, the custom input consists of a ligand that is an antibody. As used herein, the term “antibody” refers to a polypeptide of the immunoglobulin family or a polypeptide comprising fragments of an immunoglobulin that is capable of non-covalently, reversibly, and in a specific manner binding to an epitope of a corresponding antigen. The term includes, but is not limited to, polyclonal or monoclonal antibodies of the isotype classes IgA, IgD, IgE, IgG, and IgM, derived from human or other mammalian cells, including natural or genetically modified forms such as humanized, human, single-chain, chimeric, synthetic, recombinant, hybrid, mutated, grafted, and in vitro generated antibodies. The term encompasses conjugates, including but not limited to fusion proteins containing an immunoglobulin moiety, e.g., chimeric or bispecific antibodies, and fragments such as Fab, F(ab′)2, Fv, scFv, Fd, dAb and other compositions. The controller domain of the valency controllable receptor polypeptide can be configured, designed, or selected to recognize, for example, nivolumab, ipilimumab, or tocilizumab. In some embodiments, the custom input includes or consists of a multimeric antibody. e.g., a dimeric antibody or a trimeric antibody. In some embodiments, the custom input includes or consists of a chemically altered antibody. In some embodiments, the custom input includes or consists of an antibody chemically conjugated to one or more small molecules, peptides, oligonucleotides, polysaccharides, and/or lipids. In some embodiments, the custom input includes or consists of an antibody genetically modified to be linked with one or more natural or synthetic proteins or nanobodies.

In some embodiments, the custom input includes a valency control ligand that is a nanobody. In some embodiments, the custom input consists of a ligand that is a nanobody. As used herein, the terms “nanobody” or “single-domain antibody” refer to an antibody fragment comprised of a single monomeric variable antibody domain, having a molecular weight of less than 20 kDa, and able to bind selectively to a specific antigen. In some embodiments, the custom input includes or consists of a multimeric nanobody, e.g., a dimeric nanobody or a trimeric nanobody. In some embodiments, the custom input includes or consists of a chemically altered nanobody. In some embodiments, the custom input includes or consists of a nanobody chemically conjugated to one or more small molecules, peptides, oligonucleotides, polysaccharides, and/or lipids. In some embodiments, the custom input includes or consists of a nanobody genetically modified to be linked with one or more natural or synthetic proteins or antibodies.

In some embodiments, the custom input includes a valency control ligand that is a single-chain variable fragment (scFv). In some embodiments, the custom input consists of a ligand that is an scFv. As used herein, the terms “single-chain variable fragment” and “scFv” refer to an antibody in which the variable domains of the heavy chain and of the light chain of a traditional two chain antibody have been joined to form one chain. The terms can also refer to antibodies in which a linker peptide is inserted between the heavy and light chains to allow for proper folding of the single chain and creation of an active binding site. In some embodiments, the controller domain is configured, designed, or selected to recognize a valency control ligand that is an scFV that targets a tumor expressed antigen. The controller domain of the valency controllable receptor polypeptide can be configured, designed, or selected to recognize, for example, an scFV for CD19, HER2, CD22, GUCY2C, CD5, CD7, BCMA, Mesothelin, GD2, CD30, GOC3, CD123, CD20, EGFR, EGFRvIII, CEA, EpCAM, or MUC1. In some embodiments, the custom input includes or consists of a chemically altered scFv. In some embodiments, the custom input includes or consists of an ScFV chemically conjugated to one or more small molecules, peptides, oligonucleotides, polysaccharides, and/or lipids. In some embodiments, the custom input includes or consists of an scFV genetically modified to be linked with one or more natural or synthetic proteins or nanobodies.

In some embodiments, the valency control ligand is a monovalent ligand configured, designed, or selected such that one valency control ligand binds to one valency controller domain of one valency controllable receptor polypeptide. In some embodiments, the valency control ligand is a multivalent ligand configured, designed, or selected such that one valency control ligand binds to two or more, e.g., three or more, four or more, five or more, six or more, seven or more, eight or more, nine or more, or ten or more valency controller domains of different valency controllable receptor polypeptides. The valency controller domains bound by a multivalent valency control ligand can be identical or can be different from one another. By binding to multiple target controller domains in this way, a multivalent ligand can induce homo-oligomerization of two or more identical valency controllable receptor polypeptides, or can induce hetero-oligomerization of two or more different valency controllable receptor polypeptides.

In some embodiments, the custom input includes or consists of a change in an environmental parameter, e.g., a change in the extramembrane or intramembrane environment proximate to the provided valency controllable receptor polypeptide. For example, in some embodiments, the custom input includes or consists of a change in temperature, e.g., a change in the temperature of the extramembrane or intramembrane environment proximate to the valency controllable receptor polypeptide. The custom input can include or consist of an increase in the extramembrane or intramembrane temperature that is at least as large as a predetermined amount. The custom input can include or consist of a decrease in the extramembrane or intramembrane temperature that is at least as large as a predetermined amount. In some embodiments, the custom input includes or consists of a change in pH, e.g., a change in the pH of the extramembrane or intramembrane environment proximate to the valency controllable receptor polypeptide. The custom input can include or consist of an increase in the extramembrane or intramembrane pH that is at least as large as a predetermined amount. The custom input can include or consists of a decrease in the extramembrane or intramembrane pH that is at least as large as a predetermined amount.

In some embodiments, the custom input includes or consists of a change in sound, e.g., a change in the frequency, amplitude, envelope, or other property of extramembrane or intramembrane sound impinging on the valency controllable receptor polypeptide. The custom input can include or consist of sound, e.g., acoustic sound, ultrasound, and/or infrasound, directed at the receptor polypeptide. In some embodiments, the custom input includes or consists of a change in electromagnetic radiation, e.g., a change in the frequency, amplitude, or other property of extramembrane or intramembrane electromagnetic radiation impinging on the valency controllable receptor polypeptide. The custom input can include or consist of electromagnetic radiation, e.g., visible light, infrared light, ultraviolet light, X-rays, radio waves, and/or gamma rays, directed at the receptor polypeptide. In some embodiments, the custom input includes or consists of a change in mechanical force impinging on the valency controllable receptor polypeptide. The custom input can include or consist of a mechanical force directed at the receptor polypeptide.

In some embodiments, the controller domain of the provided valency controllable receptor polypeptide includes an FK506 (FKBP) binding protein family domain, or a variant or fragment thereof. In some embodiments, the controller domain consists of an FKBP binding family domain, or a variant or fragment thereof. The valency controllable receptor polypeptide controller domain can include or consist of a homodimer FKBP domain. The valency controllable receptor polypeptide controller domain can include or consist of a heterodimer FKBP/FRB domain. As used herein, the terms “variant.” and “fragment,” refer to a polypeptide related to a wild-type polypeptide, for example, either by amino acid sequence, structure (e.g., secondary and/or tertiary), activity (e.g., enzymatic activity) and/or function. Variants and fragments of a polypeptide can include one or more amino acid variations (e.g., mutations, insertions, and deletions), truncations, modifications, or combinations thereof compared to a wild-type polypeptide. A variant or fragment can include at 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the sequence, structure, activity, and/or function of the corresponding wild-type polypeptide.

In some embodiments, the controller domain of the provided valency controllable receptor polypeptide includes a bromodomain and extra terminal domain (BET) family domain, or a variant or fragment thereof. In some embodiments, the controller domain consists of a BET family domain, or a variant or fragment thereof. BET family domains suitable for use with the controller domain of the valency controllable receptor polypeptide include, for example, bromodomain-containing protein 2 (BRD2), BRD3, BRD4, and bromodomain testis-specific protein (BRDT).

In some embodiments, the controller domain of the provided valency controllable receptor polypeptide includes a B-cell lymphoma 2 (Bcl-2) family domain, or a variant or fragment thereof. In some embodiments, the controller domain consists of a Bcl-2 family domain, or a variant or fragment thereof. Bcl-2 family domains suitable for use with the controller domain of the valency controllable receptor polypeptide include, for example, Bcl-XL, Bcl-2-like 1 (BCL2L1), BCL2L2, BCL2L10, BCL2L13, BCL2L14, Bcl-2 related ovarian killer (BOK), induced myeloid leukemia cell differentiation protein Mcl-1, Mcl-2, Bim, Bid, BAD, cell death abnormality gene 9 (CED-9), Bcl-2-related protein A 1, Bfl-1, Bcl-2-associated X protein (Bax), Bcl-2 homologous antagonist/killer (Bak), Diva, Bcl-Xs, and Egl-1.

In some embodiments, the controller domain of the provided valency controllable receptor polypeptide includes a soluble N-ethylmaleimide-sensitive factor (NSF) attachment protein (SNAP) receptor domain, or a variant or fragment thereof. In some embodiments, the controller domain consists of a SNAP receptor domain, or a variant or fragment thereof. SNAP receptor domains suitable for use with the controller domain of the valency controllable receptor polypeptide include, for example, SNAP-TAG®, CLIP-TAG®, ACP-TAG, and MCP-TAG®. The controller domain can work similarly with other small molecules plus protein via covalent bond formation. In some embodiments, the controller domain includes a biotin binding domain.

In some embodiments, the extramembrane signal recognition domain of the provided valency controllable receptor polypeptide is from a naturally occurring, mutated, or synthetic receptor. The extramembrane signal receptor recognition domain can be, for example, one known by those of skill for its ability to recognize a cognate signal. In such cases, the combination of the receptor with the provided valency controller module in the valency controllable receptor polypeptide can increase the ability of the valency controllable receptor polypeptide to recognize the cognate signal. The advantageous result is that the improved valency controllable receptor polypeptide can recognize and respond to a lower concentration of the cognate signal than can a corresponding receptor polypeptide that includes the extramembrane signal recognition domain but does not include the valency controller module.

In some embodiments, the extramembrane signal recognition domain of the provided valency controllable receptor polypeptide includes a CAR. The extramembrane signal recognition domain can include or consist of a CAR, e.g., a CAR including an antigen-binding scFv or nanobody. The extramembrane signal recognition domain can include or consist of a CAR including a tumor targeting peptide sequence, e.g., a CAR having a chlorotoxin (CLTX) peptide sequence. The extramembrane signal recognition domain can include or consist of a CAR including the extramembrane domain of a natural or modified receptor, the extramembrane domain of a natural or modified membrane bound ligand, or the natural or modified soluble ligand of a tumor antigen, e.g., a CAR having an extramembrane-tethered soluble ligand IL-13 modified at a single site (E13Y). The extramembrane signal recognition domain can include or consist of a “switchable” or “universal” CAR, e.g., a SNAP-CAR or Biotin-CAR, having a small molecule protein binding domains. For example, the extramembrane signal recognition domain can include or consist of a CAR, e.g., an FITC-CAR, having a small molecule binding scFv. As another example, the extramembrane signal recognition domain can include or consist of a CAR, e.g., a PNE-CAR, having a peptide or neo-epitope binding scFv. As another example, the extramembrane signal recognition domain can include or consist of a CAR, e.g., a SUPRA-CAR, having a leucine zipper peptide sequence. The extramembrane signal recognition domain can include or consist of a CAR targeting, for example, CD19, BCMA, Mesothelin, GD2, CD30, GPC3, CD22, HER2, CD123, CD20, EGFR, EGFRvIII, CD22, CEA, EpCAM, or MUC1.

In some embodiments, the transmembrane domain of the valency controller module in the provided valency controllable receptor polypeptide includes or consists of a TLR family domain, or a variant or fragment thereof. In some embodiments, the transmembrane domain of the valency controllable receptor polypeptide includes or consists of an RTK family domain, e.g., an ErbB receptor family domain, or a variant or fragment thereof. In some embodiments, the transmembrane domain of the valency controllable receptor polypeptide includes or consists of a type I cytokine receptor family domain, or a variant or fragment thereof. In some embodiments, the transmembrane domain of the valency controllable receptor polypeptide includes or consists of a type II cytokine receptor family domain, or a variant or fragment thereof. In some embodiments, the transmembrane domain of the valency controllable receptor polypeptide includes or consists of a TGFβ receptor family domain, or a variant or fragment thereof. In some embodiments, the transmembrane domain of the valency controllable receptor polypeptide includes or consists of a TNF receptor family domain, or a variant or fragment thereof. In some embodiments, the transmembrane domain of the valency controllable receptor polypeptide includes or consists of an IgSF domain, or a variant or fragment thereof. In some embodiments, the transmembrane domain of the valency controllable receptor polypeptide includes or consists of a trk family domain, or a variant or fragment thereof. In some embodiments, the transmembrane domain of the valency controllable receptor polypeptide includes or consists of a GDNF receptor family domain, or a variant or fragment thereof.

The provided valency controllable receptor polypeptide can optionally include one or more linker units. In some embodiments, the valency controllable receptor polypeptide includes two linker units. In some embodiments, the valency controllable receptor polypeptide includes more than two linker units.

Linker units suitable for use with the provided valency controllable receptor polypeptide include, for example, those consisting of glycine (G) and serine (S). In some embodiments, at least one of the one or more linker units of the provided valency controllable receptor polypeptide is a GGS linker sequence. In some embodiments, each of the one or more linker units is GGS. In some embodiments, at least one of the one or more linker units of the valency controllable receptor polypeptide is a GGSGGSGGS linker sequence. In some embodiments, each of the one or more linker units is GGSGGSGGS. In some embodiments, at least one of the one or more linker units of the valency controllable receptor polypeptide is a GS linker sequence. In some embodiments, each of the one or more linker units is GS. In some embodiments, at least one of the one or more linker units of the valency controllable receptor polypeptide is a GSGSGS linker sequence. In some embodiments, each of the one or more linker units is GSGSGS. In some embodiments, at least one of the one or more linker units of the valency controllable receptor polypeptide is a GGGGS linker sequence, e.g., GGGGS or GGGGSGGGGSGGGGSGGGGS. In some embodiments, each of the one or more linker units is a GGGGS linker sequence. In some embodiments, at least one ofthe one or more linker units of the valency controllable receptor polypeptide is an EAAK linker sequence, e.g., EAAAK or EAAAKEAAAKEAAAK or EAAAKEAAAKEAAAKEAAAK. In some embodiments, each of the one or more linker units is an EAAAK linker sequence.

Other linker units suitable for use with the provided valency controllable receptor polypeptide include, for example. IgG hinge linker units. In some embodiments, at least one of the one or more linker units of the valency controllable receptor polypeptide is a wild-type IgG4 ESKYGPPCPPCP linker sequence. In some embodiments, each of the one or more linker units is ESKYGPPCPPCP. In some embodiments, at least one of the one or more linker units of the valency controllable receptor polypeptide is a mutated IgG4 ESKYGPPAPPAP linker sequence. In some embodiments, each of the one or more linker units is ESKYGPPAPPAP.

In some embodiments, at least one of the one or more linker units of the provided valency controllable receptor polypeptide includes an oligomerization peptide sequence or a variant or fragment thereof. In some embodiments, at least one of the one or more linker units includes a pro-clustering peptide sequence or a variant or fragment thereof. In some embodiments, at least one of the one or more linker units includes an anti-clustering peptide sequence or a variant or fragment thereof. In these ways, the linker peptides can introduce additional forces that influence the oligomerization or clustering of the receptor, and act as positive and/or negative gauges. Exemplary sequences that can influence the receptor oligomerization or clustering include those capable of forming cysteine-cysteine disulfide bonds, charge-charge-based attractions, or charge-charge-based repulsions.

In some embodiments, the valency controller domain, transmembrane domain, and optional hinge domains and linker units of the valency controller module are positioned relative to one another within the valency controllable receptor polypeptide such that the valency controller domain is located on the intramembrane side of the transmembrane domain and is therefore capable of recognizing an intramembrane custom input. For example, and as shown in FIG. 8, the valency controllable receptor polypeptide can have a structure of: [extramembrane signal recognition domain]-[hinge domain]-[transmembrane domain]-[linker unit]-[controller domain]-[intramembrane signal domain]. In some embodiments, the valency controller domain, transmembrane domain, and optional hinge domains and linker units of the valency controller module are positioned relative to one another within the valency controllable receptor polypeptide such that the valency controller domain is located on the extramembrane side of the transmembrane domain and is therefore capable of recognizing an extramembrane custom input. For example, and as shown in FIG. 9, the valency controllable receptor polypeptide can have a structure of: [extramembrane signal recognition domain]-[linker unit]-[controller domain]-[hinge domain]-[transmembrane domain]-[intramembrane signal domain].

In some embodiments, the intramembrane signaling domains of the provided valency controllable receptor polypeptide include or consist of an immunoreceptor tyrosine-based activation motifs (ITAM), or variants or fragments thereof. Immunoreceptor tyrosine-based activation motifs suitable for use with the intracellular signaling domains of the valency controllable receptor polypeptide include those of, for example, a CD3γ domain, a CD3δ domain, a CD3eε domain, a DAP12 domain, a CD32A domain, a CD32C domain, a CD132 domain, a CD79a domain, a CD79b domain, or an FCER1G.

In some embodiments, the intramembrane signaling domains of the provided valency controllable receptor polypeptide include or consist of immunoglobulin superfamily (IgSF) domains, or variants or fragments thereof. IgSF domains suitable for use with the intracellular signaling domains of the valency controllable receptor polypeptide include, for example, domains from antigen receptors such as IgA, IgD, IgE, IgG, IgM, and T-cell receptor chains. Suitable IgSF domains include those from, for example, antigen presenting molecules such as class I major histocompatibility complex (MHC), class II MHC, and β-2 microglobulin. Suitable IgSF domains include those from, for example, co-receptors such as CD4, CD8, and CD19. Suitable IgSF domains include those from, for example, antigen receptor accessory molecules such as CD3, CD79a, and CD79b. Suitable IgSF domains include those from, for example, co-stimulatory or inhibitory molecules such as CD28, CD80, and CD86. Suitable IgSF domains include, for example, killer-cell immunoglobulin-like receptors (KIR) such as KIR2DL1, KIR2DL2, KIR2DL3, KIR2DL4, KIR2DL5A, KIR2DL5B, KIR3DL1, KIR3DL2, and KIR3DL3. Suitable IgSF domains include those from, for example, leukocyte immunoglobulin-like receptors (LIRR) such as LILRB1 and LILRB2. Suitable IgSF domains include, for example, cell adhesion molecules (CAMs) such as neural cell adhesion molecules (NCAMs), intercellular adhesion molecule-1 (ICAM-1), and CD2. Suitable IgSF domains include, for example, those from growth factor receptors such as platelet-derived growth factor receptor (PDGFR) and mast/stem cell growth factor receptor precursor (SCFR). Suitable IgSF domains include, for example, those from receptor tyrosine kinases/phosphatases such as tyrosine-protein kinase receptor Tie-1 precursor, type Ila receptor protein tyrosine phosphatases (RPTPs), and type IIb RPTPs. Suitable IgSF domains include, for example, those from Ig binding receptors such as polymeric immunoglobulin receptor (PIGR). Suitable IgSF domains include, for example, those from cytoskeleton molecules such as myotilin, myopalladin, paladin, titin, obscurin, myomesin-1, and myomesin-2. Suitable IgSF domains include, for example, CD147, CD90, CD7, and butyrophilins.

In some embodiments, the intramembrane signaling domains of the provided valency controllable receptor polypeptide include or consist of a tumor necrosis factor (TNF) receptor family domain, or a variant or fragment thereof. TNF receptor family domains suitable for use with the intracellular signaling domains of the valency controllable receptor polypeptide include, for example, domains from TNF receptor superfamily 1A (TNFRSF1A, also referred to as CD120a), THFRSF1B (also referred to as CD120b), TNFRSF2 (also referred to as tumor necrosis factor or TNFα), TNFRSF3 (also referred to as lymphotoxin beta or TNFγ), TNFRSF4 (also referred to as OX40 ligand, CD252, or CD134L), TNFRSF5 (also referred to as CD40 ligand or CD154), TNFRSF6 (also referred to as Fas ligand, CD178, or CD95L), TNFRSF7 (also referred to as CD27 ligand or CD70), TNFRSF8 (also referred to as CD30 ligand or CD153), TNFRSF9 (also referred to as CD137 ligand or 4-1 BBL), TNFRSF10 (also referred to as TRAIL or CD243), TNFRSF1 (also referred to as RANKL or CD254), TNFRSF12 (also referred to as TWEAK), TNFRSF13 (also referred to as APRIL or CD256), TNFRSF13b (also referred to as BAFF or CD257), TNFRSF14 (also referred to as LIGHT or CD258), TNFRSF15 (also referred to as VEGI), TNFRSF18, and TNFRSF19 (also referred to as ectodysplasin A).

In some embodiments, the intramembrane signaling domains of the provided valency controllable receptor polypeptide include or consist of a Toll-like receptor (TLR) family domain, or a variant or fragment thereof. TLR family domains suitable for use with the intramembrane signaling domains of the valency controllable receptor polypeptide include, for example, domains from human TLR1, human TLR2, human TLR3, human TLR4, human TLR5, human TLR6, human TLR7, human TLR8, human TLR9, and human TLR10. The TLR family domain of the valency controllable receptor polypeptide intramembrane signaling domains can be, for example, a domain from murine TLR1, murine TLR2, murine TLR3, murine TLR4, murine TLR5, murine TLR6, murine TLR7, murine TLR8, murine TLR9, murine TLR11 murine TLR12, or murine TLR13. The TLR family domain of the valency controllable receptor polypeptide intramembrane domains can be from an invertebrate TLR.

In some embodiments, the intramembrane signaling domains of the valency controllable receptor polypeptide include or consist of an RTK family domain, e.g., an ErbB receptor family domain, or a variant or fragment thereof. In some embodiments, the intramembrane signaling domains of the valency controllable receptor polypeptide include or consist of a type I cytokine receptor family domain, or a variant or fragment thereof. In some embodiments, the intramembrane signaling domains of the valency controllable receptor polypeptide include or consist of a type II cytokine receptor family domain, or a variant or fragment thereof. In some embodiments, the intramembrane signaling domains of the valency controllable receptor polypeptide include or consist of a TGFβ receptor family domain, or a variant or fragment thereof. In some embodiments, the intramembrane signaling domains of the valency controllable receptor polypeptide include or consist of a trk family domain, or a variant or fragment thereof. In some embodiments, the intramembrane signaling domains of the valency controllable receptor polypeptide include or consist of a GDNF receptor family domain, or a variant or fragment thereof.

In some embodiments, the intramembrane signaling domains of the provided valency controllable receptor polypeptide include or consist of a gene editing nuclease domain, or a variant or fragment thereof. Gene editing domains suitable for use with the intramembrane signaling domains of the valency controllable receptor polypeptide include, for example, clustered regularly interspaced short palindromic repeats (CRISPR), Zinc finger nuclease, transcription activator-like effector nuclease (TALEN), CRISPR associated protein 9 (Cas9), Cas12, Cas13, Cas14, CasX, CasY, Casφ, base editor, and primer editor. In some embodiments, the intramembrane signaling domains include or consist of an epigenetic editor domain, or a variant or fragment thereof.

In some embodiments, the intramembrane signaling domains of the provided valency controllable receptor polypeptide include or consist of a transcriptional controller domain, or a variant of fragment thereof. Transcriptional controller domains suitable for use with the intramembrane signaling domains of the valency controllable receptor polypeptide include, for example, CRISPRi/a, Zinc finger, TALE, dCas9, dCas12, dCas13, dCas14, dCasX, dCasY, and dCasφ. The transcriptional controller domain can be used itself or fused to many other transcriptional or epigenetic effector domains.

In some embodiments, the intramembrane signaling domains of the provided valency controllable receptor polypeptide include or consist of an RNA controller domain, or a variant of fragment thereof. RNA controller domains suitable for use with the intramembrane signaling domains of the valency controllable receptor polypeptide include, for example, Cas13, Cas14, CasΦ, ADARs, Argonaute, and engineered variants thereof.

In some embodiments, the intramembrane signaling domains of the provided valency controllable receptor polypeptide include or consist of a protein controller domain, or a variant of fragment thereof. Protein controller domains suitable for use with the intramembrane signaling domains of the valency controllable receptor polypeptide include, for example, a protease such as Tobacco Etch Virus (TEV) protease, hepatitis C virus (HCV) protease, and human immunodeficiency virus-1 (HIV-1) protease. Protein controller domains suitable for use with the intramembrane signaling domains include, for example, antiCRISPRs (Acrs) and other post-modification enzymes and degradation complexes.

Other intramembrane signaling domains suitable for use with the provided valency controllable receptor polypeptide include, but are not limited to, 2B4, BTLA, CD2, CD22, CD4, CD84, CD8a, CD8b, CRACC, CRTAM, CTLA-4. DAP10, DAP12, DNAM-1, DR3, FCER1G, FCGR1A, FCGR2B, FCGR3A, FCRL1, FCRL2, FCRL3, FCRL4, FCRL5, FCRL6, GITR, HVEM, ICOS, LAG3, LAT, Ly9, NKG2A, NKG2C, NKG2D, NKp30, NKp44, NKp46, PD-1, PILRB, SIRPa, SLAMF1, SLAMF6, SLAMF7, TIGIT, TIMI, TIM3, CD300a, CD300f, CD3g, CD3e, CD3d, CD3z, CD200R, CD244, CD72, CD96, CXADR, DC-SIGN, ICOS, KLRG1, LAIRI, KLRB1, NTB-A, Siglec-3, and TAC.

The one or more intramembrane signaling domains of the provided valency controllable receptor polypeptide are each independently configured to induce the modulation of one or more intramembrane pathways upon oligomerization, e.g., dimerization or 3-dimensional (3D) clustering, of the receptor polypeptide. In some embodiments, the intramembrane signaling domains are configured to modulate two intramembrane signal pathways. In some embodiments, the intramembrane signaling domains modulate more than two pathways, e.g., more than three pathways, more than four pathways, more than five pathways, more than six pathways, more than seven pathways, more than eight pathways, more than nine pathways, or more than ten pathways.

In some embodiments, at least one of the one or more intramembrane pathways modulated by the intramembrane signaling domains is activated by the intramembrane signaling domains. In some embodiments, each of the one or more intramembrane pathways modulated by the intramembrane signaling domains is activated by the intramembrane signaling domains. In some embodiments, at least one of the one or more intramembrane pathways modulated by the intramembrane signaling domains is inhibited by the intramembrane signaling domains. In some embodiments, each of the one or more intramembrane pathways modulated by the intramembrane signaling domains is inhibited by the intramembrane signaling domains. In some embodiments, at least one of the one or more intramembrane pathways modulated by the intramembrane signaling domains is enhanced by the intramembrane signaling domains. In some embodiments, each of the one or more intramembrane pathways modulated by the intramembrane signaling domains is enhanced by the intramembrane signaling domains.

In some embodiments, at least one of the one or more intramembrane pathways modulated by the intramembrane signaling domain is an exogenous pathway. In some embodiments, each of the one or more intramembrane pathways modulated by the intramembrane signaling domains is an exogenous pathway. In some embodiments, at least one of the one or more intramembrane pathways modulated by the intramembrane signaling domains is an endogenous pathway. In some embodiments, each of the one or more intramembrane pathways modulated by the intramembrane signaling domains is an endogenous pathway.

In some embodiments, at least one of the one or more intramembrane pathways modulated by the intramembrane signaling domains is a synthetic pathway. In some embodiments, each of the one or more intramembrane pathways modulated by the intramembrane signaling domains is a synthetic pathway. In some embodiments, at least one of the one or more intramembrane pathways modulated by the intramembrane signaling domains is a naturally occurring pathway. In some embodiments, each of the one or more intramembrane pathways modulated by the intramembrane signaling domains is a naturally occurring pathway.

A wide variety of intramembrane pathways are suitable for modulation by a valency controllable receptor polypeptide as disclosed herein. The one or more intramembrane pathways modulated by the valency controllable receptor polypeptide can include, for example, pathways responsible for genome sequence editing, transcription activation or repression, epigenetic modifications, genome translocation and rearrangement, RNA expression or degradation, RNA splicing or processing, post-transcription modifications of mRNA or ncRNA, post-translational modifications of proteins, cleavage or proteolysis of proteins, production or degradation of metabolites or other chemistries, antigen recognition or binding, trafficking of signaling molecules, cell cycle control, cell differentiation or reprogramming, T cell activation or exhaustion, programmed cell death, cell trafficking, secretion of cytokines or hormones, neuronal activity, macrophage phagocytosis, neutrophil NETpoptosis, immunological synapse formation, myeloid cell degranulation, antigen presentation, secretion or hypermutation of antibodies, and/or production of oncolytic virus.

### Valency Controllable Receptor Systems

In another aspect, a valency controllable receptor system is disclosed. The system includes two or more valency controllable receptor polypeptides, at least one of which is a valency controllable receptor polypeptide having a structure as described above. Each of the other valency controllable receptor polypeptides of the valency controllable receptor system can optionally also have a structure as described above, or can alternatively have a structure as described above but lacking an extramembrane signal recognition domain. In this way, each of the valency controllable receptor polypeptides of the valency controllable receptor system will have the capability of oligomerizing through the function of the valency controller module of the polypeptide, and each will also have the capability of transmitting intramembrane signals through the function of the intramembrane signaling domains of the polypeptide. Further, at least one of valency controllable receptor polypeptides will also have the capability of recognizing an extramembrane signal through the function of the extramembrane signal recognition domain of the polypeptide. The valency controllable receptor system can include two, three, four, five, six, seven, eight, nine, ten, or more than ten valency controllable receptor polypeptides.

In some embodiments, each of the valency controllable receptor polypeptides of the valency controllable receptor system is identical to one another. In some embodiments, at least one of the valency controllable receptor polypeptides of the valency controllable receptor system is different from another valency controllable receptor polypeptide of the system. The differences between the valency controllable receptor polypeptides can include, for example, different extramembrane signal recognition domains. For example, one of the valency controllable receptor polypeptides can include an extramembrane signal recognition domain while another does not. Alternatively, one of the valency controllable receptor polypeptides can include an extramembrane signal recognition domain that is different from an extramembrane signal recognition domain of the other valency controllable receptor polypeptide. The differences between the valency controllable receptor polypeptides can include, for example, different valency controller modules. For example, one of the valency controller modules can recognize a first custom input, while the other valency controller module recognizes a different second custom input. Alternatively, two different valency controller modules can recognize the same custom input, e.g., a multivalent valency control ligand. The differences between the valency controllable receptor polypeptides can include, for example, different intramembrane signal domains. For example, each intramembrane signal domain of one of the valency controllable receptor polypeptides can differ from each intramembrane signal domain of another of the valency controllable receptor polypeptides. Alternatively, at least one intramembrane signal domain of a first of the valency controllable receptor polypeptides can differ from at least one intramembrane signal domain a second of the valency controllable receptor polypeptides, while at least one other intramembrane signal domain of the first valency controllable receptor polypeptides is identical to at least one other intramembrane signal domain of the second valency controllable receptor polypeptide.

The valency controllable receptor system can further include a membrane, such that the transmembrane domains of the valency controllable receptor polypeptides of the system are located within the membrane. The membrane of the system separates two spatial regions, which can be, for example, an extracellular and intracellular region, a cytoplasmic and nuclear region, or regions outside and inside of an intracellular vesicle or organelle. The valency controllable receptor polypeptides are situated such that for each polypeptide the extramembrane domain and the intramembrane domain are located on opposite sides of the membrane, while the intramembrane domain is located within the membrane. In some embodiments, the membrane of the provided system is a cellular membrane. In some embodiments, the membrane of the provided system is a nuclear membrane. In some embodiments, the membrane of the provided system is an organelle membrane. In some embodiments, the membrane of the provided system is a vesicle membrane.

The valency controllable receptor system can further include a valency control ligand that is the custom input recognized by at least one valency controller domain of at least one valency controllable receptor polypeptide of the system. In some embodiments, the valency controllable receptor system includes two or more valency control ligands that are each independently a custom input recognized by at least one valency controller domain of at least one valency controllable receptor polypeptide of the system. The valency controllable receptor system can further include an antigen that is the extramembrane signal recognized by at least one extramembrane signal recognition domain of at least one valency controllable receptor polypeptide of the system. In some embodiments, the valency controllable receptor system includes two or more antigens that are each independently an extramembrane signal recognized by at least one extramembrane signal recognition domain of at least one valency controllable receptor polypeptide of the system.

### Host Cells

In another aspect, the disclosure provides a host cell including any of the valency controllable receptor polypeptides or systems described herein. In some embodiments, the host cell expresses one or more of the provided valency controllable receptor polypeptides. As used herein, the term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton. cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens, and the like), seaweeds (e.g., kelp), a fungal cell (e.g., a yeast cell, a cell from a mushroom), a cell from an invertebrate animal (e.g., fruit fly, cnidarian, echinoderm, nematode, mollusk, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), etc. Sometimes a cell does not originate from a natural organism (e.g., a cell can be a synthetically made, sometimes termed an artificial cell).

A wide variety of cell types are suitable for use as the provided host cell. In some embodiments, the host cell is an immune cell, including any cell that is involved in an immune response. For example, the use of engineered innate immune cells such as optionally allogenic natural NK cells, iPSC-derived NK cells, and/or macrophage cells can greatly facilitate treatment of solid tumor while avoiding side effects. This allows ligand-mediated precise activation of immunity at a precise location (e.g., presence of a local tumor signal) and at a specific time point (e.g., administration of the drug). This effect is inducible and reversible.

In some embodiments, the host immune cell type includes granulocytes such as basophils, eosinophils, and neutrophils; mast cells; phagocytes such as monocytes which can develop into macrophages; antigen-presenting cells such as dendritic cells; and lymphocytes such as natural killer cells (NK cells), B cells, T cells, and innate lymphoid cells (ILC). In some embodiments, the host immune cell is an immune effector cell. An immune effector cell is an immune cell that can perform a specific function in response to a stimulus. In some embodiments, the host immune cell is an immune effector cell which can induce cell death. In some embodiments, the host immune cell is a lymphocyte. In some embodiments, the lymphocyte is a NK cell. In some embodiments the lymphocyte is a T cell. In some embodiments, the T cell is an activated T cell. T cells include both naive and memory cells (e.g., central memory or T_{CM}, effector memory or T_{EM} and effector memory RA or T_{EMRA}), effector cells (e.g., cytotoxic T cells or CTLs or Tc cells), helper cells (e.g., Th1, Th2, Th3, Th9, Th7, TFH), regulatory cells (e.g., Treg, and Trl cells), natural killer T cells (NKT cells), tumor infiltrating lymphocytes (TILs), lymphocyte-activated killer cells (LAKs), αβ T cells, γδ T cells, and similar unique classes of the T cell lineage.

T cells can be divided into two broad categories: CD8+ T cells and CD4+ T cells, based on which protein is present on the cell's surface. T cells expressing a provided valency controllable receptor polypeptide can carry out multiple functions, including killing infected cells and activating or recruiting other immune cells. CD8+ T cells are referred to as cytotoxic T cells or cytotoxic T lymphocytes (CTLs). CTLs expressing a provided valency controllable receptor polypeptide can be involved in recognizing and removing virus-infected cells and cancer cells. CTLs have specialized compartments, or granules, containing cytotoxins that cause apoptosis, e.g., programmed cell death. CD4+ T cells can be subdivided into four sub-sets—Th1, Th2, Th17, and Treg, with “Th” referring to “T helper cell,” although additional sub-sets may exist. Th1 cells can coordinate immune responses against intracellular microbes, especially bacteria. They can produce and secrete molecules that alert and activate other immune cells, like bacteria-ingesting macrophages. Th2 cells are involved in coordinating immune responses against extracellular pathogens, like helminths (parasitic worms), by alerting B cells, granulocytes, and mast cells. Th17 cells can produce interleukin 17 (IL-17), a signaling molecule that activates immune and non-immune cells. Th17 cells are important for recruiting neutrophils.

The receptor can be engineered into regenerative host cell types to direct the ligand-mediated cell differentiation and programming. This allows site-specific differentiation of cells and tissues to repair or regenerate a damaged or aged body. In some embodiments, the host cell is a stem cell. The host cell can be, for example, an induced pluripotent stem cell (iPSC), an embryonic stem cell (ESC), an adult stem cell, or a mesenchymal stem cell (MSC). In some embodiments, the host cell is a progenitor cell. The host cell can be, for example, a neural progenitor cell, a skeletal progenitor cell, a muscle progenitor cell, a fat progenitor cell, a heart progenitor cell, a chondrocyte, a fibroblast cell, or a pancreatic progenitor cell. The host cell can be, for example, a progenitor-differentiated cell, a stem cell-differentiated cell, an organoid, or an assembloid.

In another aspect, a population of host cells is provided. Each host cell of the population independently includes a valency controllable receptor as disclosed herein, or a valency controllable receptor system as disclosed herein.

### Methods for Modulating Pathways

In another aspect, a method for modulating, e.g., activating, inhibiting, or enhancing, an intramembrane signal pathway is disclosed. The modulated intramembrane signal pathway can be any of those disclosed herein. The intramembrane signal pathway modulation method includes providing any of the valency controllable receptor polypeptides as disclosed herein, any of the valency controllable receptor systems as disclosed herein, any of the host cells as disclosed herein, or any of the populations of host cells as disclosed herein. In some embodiments, the method further includes exposing at least one of the provided valency controllable receptor polypeptides to the custom input recognized by the valency control module of the valency controllable receptor polypeptide. Exposing can be performed for any suitable length of time, for example at least 1 minute, at least 5 minutes, at least 10 minutes, at least 30 minutes, at least 1 hour, at least 2 hours, at least 3 hours, at least 4 hours, at least 5 hours, at least 6 hours, at least 7 hours, at least 8 hours, at least 12 hours, at least 16 hours, at least 20 hours, at least 24 hours, at least 2 days, at least 3 days, at least 4 days, at least 5 days, at least 6 days, at least 1 week, at least 2 weeks, at least 3 weeks, at least 1 month or longer.

### Methods for Disease Prevention or Treatment

In another aspect, a method for preventing or treating a disease in a subject is disclosed. The new class of synthetic receptors disclosed herein can be used according to the provided method for next generation cell therapies. By inducing the provided valency controllable receptor polypeptides with either endogenous (e.g., cytokines, tumor microenvironment signals, antigens) or exogenous (e.g., small molecule drugs, peptides, biologics, ultrasound) extramembrane or intramembrane custom inputs, and “wiring” the receptor to various cellular pathways, these engineered cell therapy methods can have indications for any malady for which cells are involved, such as cancer, infectious diseases, wound healing, autoimmunity, regenerative medicine. CNS diseases, and anti-aging.

As used herein, the term “treatment” refers to an approach for obtaining beneficial or desired results including but not limited to a therapeutic benefit and/or a prophylactic benefit. A therapeutic benefit imparts any relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested. A treatment can involve any of ameliorating one or more symptoms of disease, e.g., cancer, preventing the manifestation of such symptoms before they occur; slowing down or completely preventing the progression of the disease (as may be evident by longer periods between reoccurrence episodes, slowing down or prevention of the deterioration of symptoms, etc.), enhancing the onset of a remission period, slowing down the irreversible damage caused in the progressive-chronic stage of the disease (both in the primary and secondary stages), delaying the onset of said progressive stage, or any combination thereof.

The provided disease prevention or treatment methods include administering to the subject a therapeutically effective amount of the valency controllable receptor polypeptides as disclosed herein, the valency controllable receptor systems as disclosed herein, the host cells as disclosed herein, or the populations of host cells as disclosed herein. As used herein, the term “administering” refers to delivery of agents or compositions to the desired site of biological action. Administration methods include, but are not limited to parenteral administration (e.g., intravenous, subcutaneous, intraperitoneal, intramuscular, intravascular, intrathecal, intranasal, intravitreal, infusion and local injection), transmucosal injection, oral administration, administration as a suppository, and topical administration. Other modes of delivery include, but are not limited to, the use of liposomal formulations, intravenous infusion, transplantation, etc. One skilled in the art will know of additional methods for administering a therapeutically effective amount of a composition of the present disclosure for preventing or relieving one or more symptoms associated with a disease

As used herein, the term “therapeutically effective amount” refers to the quantity of a composition that is sufficient to result in a desired activity upon administration to a subject in need thereof. Within the context of the present disclosure, the term “therapeutically effective” refers to that quantity of a composition that is sufficient to delay the manifestation, arrest the progression, or relieve or alleviate at least one symptom of a disorder treated by the methods of the present disclosure.

Pharmaceutical compositions containing valency controllable receptor polypeptides, systems, or host cells described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Amounts effective for this use can vary based on the severity and course of the disease or condition, previous therapy, the subject's health status, weight, and response to the drugs, and the judgment of the treating physician.

Valency controllable receptor polypeptides, systems, or host cells described herein can be administered before, during, or after the occurrence of a disease or condition, and the timing of administering the composition can vary. For example, a pharmaceutical compositions can be used as a prophylactic and can be administered continuously to subjects with a propensity to conditions or diseases in order to prevent the occurrence of the disease or condition. The pharmaceutical compositions can be administered to a subject during or as soon as possible after the onset of the symptoms. The administration can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A composition can be administered as soon as is practicable after the onset of a disease or condition is detected or suspected, and for a length of time necessary for the treatment of the disease, such as, for example, from about 1 month to about 3 months. The length of treatment can vary for each subject.

A wide variety of diseases can be prevented or treated using the provided methods. The invention is suitable for broad cell therapy applications to treat, for example, blood or solid cancer, cancerous tumors, viral infections, bacterial infections, genetic diseases, wound healing, autoimmunity, regenerative medicine, CNS diseases, and anti-aging.

In some embodiments, the prevented or treated disease is a cancer. Non-limiting examples of cancers that can be treated with the provided valency controllable receptor polypeptides, systems, or host cells include Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myclomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithclioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myclodysplastic Syndromes, Mycloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Vemer Morrison syndrome, Verrucous carcinoma, Visual Pathway Glioma, Vulvar Cancer, Waldenstrom's macroglobulinemia, Warthin's tumor, Wilms' tumor, and combinations thereof.

In some embodiments, the prevented or treated disease is a cancerous tumor. The cancerous tumor can be a solid cancerous tumor or a liquid cancerous tumor. The liquid cancerous tumor can be, for example, a lymphoma or a leukemia. A tumor treated with the methods disclosed herein can result in stabilized tumor growth (e.g., one or more tumors do not increase more than 1%, 5%, 10%, 15%, or 20% in size, and/or do not metastasize). In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25%, 30%, 35%, 40%, 45%, 50%, 55%, 60%, 65%, 70%, 75%, 80%, 85%, 90%, 95% or more. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

In some embodiments, the prevented or treated disease is an infectious disease. The infectious disease can be, for example, a viral infectious disease. The infectious disease can be, for example, a bacterial infectious disease. In the case of bacterial infections, the innate immune system must recognize specific markers of the microbes in order to clear the pathogen. These pathogen associated molecular patterns are recognized by various receptors, most notably the TLRs that are common to all immune cells and work to activate immune pathways that turn on their bactericidal capacities. In the case of macrophages, recognition of microbial pathogens through a TLR activates their unique ability to engulf the bacteria within themselves and destroy the pathogen by acidification. However, bacteria have mechanisms to evade macrophages by hiding the molecules that cause this activation. For example, during biomedical device implantations such as catheters and pacemakers, bacteria form dense biofilms around themselves to avoid recognition, causing massive infection issues which have inhibited lifesaving technology from moving to the clinic. The provided valency controllable receptor polypeptide, system, or host cell can “rewire” these TLRs to recognize the constituents of the biofilm itself as opposed to the bacteria, such that macrophages are activated by the evasion mechanisms, destroying the infection and allowing these devices to be more safely implanted.

In some embodiments, the method further includes exposing the administered valency controllable receptor to the custom input. The exposing of the valency controllable receptor to the extramembrane signal can include, for example, introducing a therapeutically effective amount of the custom input to the subject.

### EMBODIMENTS

The following embodiments are contemplated. All combinations of features and embodiment are contemplated.

Embodiment 1: A valency controllable receptor polypeptide comprising: an extramembrane signal recognition domain having an ability to recognize an extramembrane signal; a valency controller module comprising: a controller domain connected to the extramembrane signal recognition domain, wherein the controller domain is configured to recognize a custom input different from the extramembrane signal; and a transmembrane domain connected to the controller domain; wherein the valency controller module is configured to oligomerize the valency controllable receptor polypeptide upon recognition of the custom input by the controller domain; and one or more intramembrane signaling domains connected to the transmembrane domain.

Embodiment 2: An embodiment of embodiment 1, wherein the valency controller module further comprises: one or more linker units connecting the one or more controller domains to the extramembrane domain.

Embodiment 3: An embodiment of embodiment 1 or 2, wherein the valency controllable module further comprises: one or more hinge domains connecting the one or more controller domains to the transmembrane domain.

Embodiment 4: An embodiment of any of the embodiments of embodiment 1-3, wherein the one or more intramembrane signaling domains each independently modulate one or more intramembrane pathways.

Embodiment 5: An embodiment of embodiment 4, wherein at least one of the intramembrane signaling domains activates one or more intramembrane pathways.

Embodiment 6: An embodiment of embodiment 4 or 5, wherein at least one of the intramembrane signaling domains inhibits one or more intramembrane pathways.

Embodiment 7: An embodiment of any of the embodiments of embodiment 4-6, wherein at least one of the intramembrane signaling domains enhances one or more intramembrane pathways.

Embodiment 8: An embodiment of any of the embodiments of embodiment 4-7, wherein at least one of the one or more intramembrane pathways is an endogenous pathway.

Embodiment 9: An embodiment of any of the embodiments of embodiment 4-8, wherein at least one of the one or more intramembrane pathways is an exogenous pathway.

Embodiment 10: An embodiment of any of the embodiments of embodiment 4-9, wherein at least one of the one or more intramembrane pathways is a synthetic pathway.

Embodiment 11: An embodiment of any of the embodiments of embodiment 4-10, wherein the one or more intramembrane pathways comprise genome sequence editing, transcription activation or repression, epigenetic modifications, genome translocation and rearrangement, RNA expression or degradation, RNA splicing or processing, post-transcription modifications of mRNA or ncRNA, post-translational modifications of proteins, cleavage or proteolysis of proteins, production or degradation of metabolites or other chemistries, antigen recognition or binding, trafficking of signaling molecules, cell cycle control, cell differentiation or reprogramming, T cell activation or exhaustion, programmed cell death, cell trafficking, secretion of cytokines or hormones, neuronal activity, macrophage phagocytosis, neutrophil NETpoptosis, immunological synapse formation, myeloid cell degranulation, antigen presentation, secretion or hypermutation of antibodies, production of oncolytic virus, or a combination thereof.

Embodiment 12: An embodiment of any of the embodiments of embodiment 1-11, wherein the custom input is an extramembrane input.

Embodiment 13: An embodiment of any of the embodiments of embodiment 1-11, wherein the custom input is an intramembrane input.

Embodiment 14: An embodiment of any of the embodiments of embodiment 1-13, wherein the custom input is a valency control ligand.

Embodiment 15: An embodiment of embodiment 14, wherein the valency control ligand comprises a small molecule drug.

Embodiment 16: An embodiment of embodiment 14 or 15, wherein the valency control ligand comprises a metabolite.

Embodiment 17: An embodiment of any of the embodiments of embodiment 14-16, wherein the valency control ligand comprises an oligonucleotide.

Embodiment 18: An embodiment of any of the embodiments of embodiment 14-17 wherein the valency control ligand comprises a peptide, a protein, a polysaccharide, a lipid, a glycoprotein, or a combination thereof.

Embodiment 19: An embodiment of any of the embodiments of embodiment 14-18 wherein the valency control ligand comprises an antibody, a nanobody, an scFv, a hormone, or a cytokine.

Embodiment 20: An embodiment of any of the embodiments of embodiment 14-19, wherein the valency control ligand comprises a polymer scaffold to which one or more interaction moieties are conjugated, wherein each of the one or more interaction moieties is independently a small molecule, a peptide, an oligonucleotide, a polysaccharide, or a lipid.

Embodiment 21: An embodiment of any of the embodiments of embodiment 1-13, wherein the custom input comprises a change in temperature or pH.

Embodiment 22: An embodiment of any of the embodiments of embodiment 1-13, wherein the custom input comprises a change in sound or electromagnetic radiation.

Embodiment 23: An embodiment of any of the embodiments of embodiment 1-13, wherein the custom input comprises a change in mechanical force.

Embodiment 24: An embodiment of any of the embodiments of embodiment 1-23, wherein the extramembrane signal recognition domain comprises a soluble N-ethylmaleimide-sensitive factor (NSF) attachment protein (SNAP) receptor domain, a biotin-binding domain, a leucine zipper domain, a single-chain variable fragment (scFv) domain, a nanobody domain, a cell-targeting domain from a natural polypeptide, or a variant or fragment thereof.

Embodiment 25: Tc valency controllable receptor polypeptide of any one of claims 1-24, wherein the controller domain comprises a small molecule binding domain.

Embodiment 26: An embodiment of any of the embodiments of embodiment 1-24, wherein the controller domain comprises an FK506 binding protein (FKBP) family domain, a bromodomain and extra terminal domain (BET) family domain, a gibberellin-insensitive dwarf (GID) family domain, a B-cell lymphoma 2 (Bcl-2) family domain, a soluble N-ethylmaleimide-sensitive factor (NSF) attachment protein (SNAP) receptor domain a biotin-binding domain, a nanobody, a single-chain variable fragment (scFv) domain, an E3 ubiquitin ligase domain, a natural or synthetic peptide epitope, or a variant or fragment thereof.

Embodiment 27: An embodiment of any of the embodiments of embodiment 1-26, wherein the one or more intramembrane signaling domains comprise a CD3ζ (CD247) domain.

Embodiment 28: An embodiment of any of the embodiments of embodiment 1-27, wherein the one or more intramembrane signaling domains comprise an immunoreceptor tyrosine-based activation motif (ITAM).

Embodiment 29: An embodiment of embodiment 28, wherein the ITAM comprises a sequence from a CD3γ domain, from a CD3S domain, from a CD3eε domain, from a DAP12 domain, from a CD32A domain, from a CD32C domain, from a CD132 domain, from a CD79a domain, from a CD79b domain, or from an FCER1G domain.

Embodiment 30: An embodiment of any of the embodiments of embodiment 1-29, wherein the one or more intramembrane signaling domains comprise a CD28 domain, a 4-1BB domain, an OX40 (CD134) domain, an ICOS (CD278) domain, a CD40 domain, a CD2 domain, or a CD27 domain.

Embodiment 31: A valency controllable receptor system comprising: two or more valency controllable receptor polypeptides, each independently a valency controllable receptor polypeptide of any of the embodiments of embodiment 1-30.

Embodiment 32: An embodiment of embodiment 31, wherein the amino acid sequence of each of the two or more valency controllable receptor polypeptides is identical.

Embodiment 33: An embodiment of embodiment 31, wherein the amino acid sequence of a first valency controllable receptor polypeptide of the two or more valency controllable receptor polypeptides differs from the amino acid sequence of a second valency controllable receptor polypeptide of the two or more valency controllable receptor polypeptides.

Embodiment 34: An embodiment of embodiment 33, wherein at least one of the one or more intramembrane signaling domains of the first valency controllable receptor polypeptide differs from at least one of the one or more intramembrane signaling domains of the second valency controllable receptor polypeptide.

Embodiment 35: An embodiment of embodiment 33 or 34, wherein the first valency controllable receptor polypeptide comprises a first controller domain configured to recognize a first custom input, and wherein the second valency controllable receptor polypeptide comprises a second controller domain configured to recognize a second custom input different from the first custom input.

Embodiment 36: An embodiment of any of the embodiments of embodiment 31-35, further comprising: a membrane, wherein the transmembrane domain of each of the two or more valency controllable receptor polypeptides is located within the membrane.

Embodiment 37: An embodiment of embodiment 36, wherein the membrane is a cellular membrane, a nuclear membrane, an organelle membrane, or a vesicle membrane.

Embodiment 38: An embodiment of any of the embodiments of embodiment 31-37, further comprising: a valency control ligand, wherein the valency control ligand is the custom input recognized by the control domain of at least one of the one or more valency control polypeptides.

Embodiment 39: An embodiment of any of the embodiments of embodiment 31-38, further comprising: an antigen, wherein the antigen is the extramembrane signal recognized by the extramembrane signal recognition domain of the one or more valency control polypeptides.

Embodiment 40: A valency controllable receptor system comprising: a first valency controllable receptor polypeptides, wherein the first valency controllable receptor polypeptide is a valency controllable receptor polypeptide of any of the embodiments of embodiment 1-30; and a second valency controllable receptor polypeptide comprising: a second valency controller module comprising: a second controller domain configured to recognize a second custom input different from the extramembrane signal; and a second transmembrane domain connected to the second controller domain; wherein the second valency controller module is configured to oligomerize the second valency controllable receptor polypeptide upon recognition of the second custom input by the second controller domain; and one or more intramembrane signaling domains connected to the second transmembrane domain.

Embodiment 41: An embodiment of embodiment 40, wherein the second valency controllable receptor polypeptide does not comprise an extramembrane signal recognition domain having an ability to recognize an extramembrane signal different from the second custom input.

Embodiment 42: An embodiment of embodiment 40 or 41, wherein the second valency controllable module is configured to oligomerize the first valency controllable receptor polypeptide with the second valency controllable receptor polypeptide upon recognition of the second custom input.

Embodiment 43: An embodiment of any of the embodiments of embodiment 40-42, wherein the second custom input is the same as the custom input recognized by the valency controller module of the first valency controllable receptor polypeptide.

Embodiment 44: An embodiment of embodiment 43, wherein the amino acid sequence of the second valency controller module is the same as the amino acid sequence of the valency controller module of the first valency controllable receptor polypeptide.

Embodiment 45: An embodiment of any of the embodiments of embodiment 4044, wherein at least one of the one or more intramembrane signaling domains of the second valency controllable receptor polypeptide differs from at least one of the one or more intramembrane signaling domains of first valency controllable receptor polypeptide.

Embodiment 46: An embodiment of any of the embodiments of embodiment 40-45, further comprising: a membrane, wherein the second transmembrane domain and the transmembrane domain of the first valency controllable receptor polypeptides are located within the membrane.

Embodiment 47: An embodiment of embodiment 46, wherein the membrane is a cellular membrane, a nuclear membrane, an organelle membrane, or a vesicle membrane.

Embodiment 48: An embodiment of any of the embodiments of embodiment 40-47, further comprising: a valency control ligand, wherein the valency control ligand is the custom input recognized by the second valency control domain or the valency control domain of the first valency controllable receptor polypeptide.

Embodiment 49: An embodiment of any of the embodiments of embodiment 4048, further comprising: an antigen, wherein the antigen is the extramembrane signal recognized by the extramembrane signal recognition domain of the first valency controllable receptor polypeptide. [01%] Embodiment 50: A host cell comprising the valency controllable receptor polypeptide of any of the embodiments of embodiment 1-30, or the valency controllable receptor system of any of the embodiments of embodiment 31-49.

Embodiment 51: An embodiment of embodiment 50, wherein the host cell is a lymphocyte, a phagocytic cell, a granulocytic cell, or a dendritic cell.

Embodiment 52: An embodiment of embodiment 51, wherein the lymphocyte is a T cell, a B cell, a natural killer (NK) cell, or an innate lymphoid cell (ILC).

Embodiment 53: An embodiment of embodiment 52, wherein the T cell is a CD4+ helper αβT cell, a CD8+ killer αβT cell, a δγT cell, or a natural killer T (NKT) cell.

Embodiment 54: An embodiment of embodiment 51, wherein the phagocytic cell is a monocyte or a macrophage.

Embodiment 55: An embodiment of embodiment 51, wherein the granulocytic cell is a neutrophil, a basophil, an eosinophil, or a mast cell.

Embodiment 56: An embodiment of embodiment 50, wherein the host cell is a stem cell or a progenitor cell.

Embodiment 57: An embodiment of embodiment 56, wherein the stem cell is an induced pluripotent stem cell (iPSC), an embryonic stem cell (ESC), an adult stem cell, or a mesenchymal stem cell (MSC).

Embodiment 58: An embodiment of embodiment 56, wherein the progenitor cell is a neural progenitor cell, a skeletal progenitor cell, a muscle progenitor cell, a fat progenitor cell, a heart progenitor cell, a chondrocyte, a fibroblast cell, or a pancreatic progenitor cell.

Embodiment 59: An embodiment of embodiment 50, wherein the host cell is a progenitor-cell-differentiated cell, a stem-cell-differentiated cell, an organoid, or an assembloid.

Embodiment 60: A method for modulating an intramembrane pathway, the method comprising providing the valency controllable receptor polypeptide of any of the embodiments of embodiment 1-30, or the valency controllable receptor system of any of the embodiments of embodiment 31-49.

Embodiment 61: An embodiment of embodiment 60, further comprising exposing the valency controllable receptor polypeptide to the custom input.

Embodiment 62: A method for preventing or treating a disease in a subject, the method comprising administering to the subject an amount of the valency controllable receptor polypeptide of any of the embodiments of embodiment 1-30, the valency controllable receptor system of any of the embodiments of embodiment 31-49, or the host cell of any of the embodiments of embodiment 50-59.

Embodiment 63: An embodiment of embodiment 62, further comprising, subsequent to the administering, exposing the valency controllable receptor polypeptide to the custom input.

Embodiment 64: An embodiment of embodiment 63, wherein the exposing comprises introducing to the subject a therapeutically effective amount of the custom input.

Embodiment 65: An embodiment of any of the embodiments of embodiment 62-64, wherein the disease is a cancer.

Embodiment 66: An embodiment of any of the embodiments of embodiment 62-64, wherein the disease is a cancerous tumor.

Embodiment 67: An embodiment of embodiment 66, wherein the cancerous tumor is a solid cancerous tumor.

Embodiment 68: An embodiment of embodiment 66, wherein the cancerous tumor is a liquid cancerous tumor.

Embodiment 69: An embodiment of any of the embodiments of embodiment 62-64, wherein the disease is an infectious disease.

Embodiment 70: An embodiment of embodiment 69, wherein the infectious disease is a viral infectious disease or a bacterial infectious disease.

Embodiment 71: An embodiment of any of the embodiments of embodiment 62-64, wherein the disease is an autoimmune disease.

Embodiment 72: An embodiment of any of the embodiments of embodiment 62-64, wherein the disease is an age-related disease.

Embodiment 73: An embodiment of any of the embodiments of embodiment 62-64, wherein the disease is a neurological disease.

Embodiment 74: A method for healing a wound in a subject, the method comprising administering to the subject an amount of the valency controllable receptor polypeptide of any of the embodiments of embodiment 1-30, the valency controllable receptor system of any of claims the embodiments of embodiment 31-49, or the host cell of any of the embodiments of embodiment 50-59.

Embodiment 75: An embodiment of embodiment 74, further comprising, subsequent to the administering, exposing the valency controllable receptor polypeptide to the custom input.

Embodiment 76: An embodiment of embodiment 75, wherein the exposing comprises introducing to the subject a therapeutically effective amount of the custom input.

### EXAMPLES

The present disclosure will be better understood in view of the following non-limiting examples. The following examples are intended for illustrative purposes only and do not limit in any way the scope of the present invention.

### Example 1. Construction of a Valency Controller Module and a Valency Controllable Receptor CAR Polypeptide

A valency controller module was constructed according to the schematic illustration of FIG. 1. The valency controller module included a controller domain configured to recognize a small molecule, where the controller domain was fused to an engineered hinge and transmembrane domain of the valency controller module. More specifically, the valency controller module included a monomeric streptavidin domain as its controller domain, where the controller domain was configured to recognize a multivalent 4-mer biotin valency control ligand. The valency controller module was inserted into a chimeric antigen receptor (CAR) polypeptide between its single chain variable fragment (scFv) and its intracellular signaling domains to establish a valency controllable receptor CAR (VCR-CAR) polypeptide. The valency controller module of the valency controllable receptor polypeptide is one configured to recognize an extracellular custom input, i.e., the 4-mer biotin ligand.

### Example 2. Construction of 2nd Generation CAR T Cells Modified with a Valency Controllable Receptor System

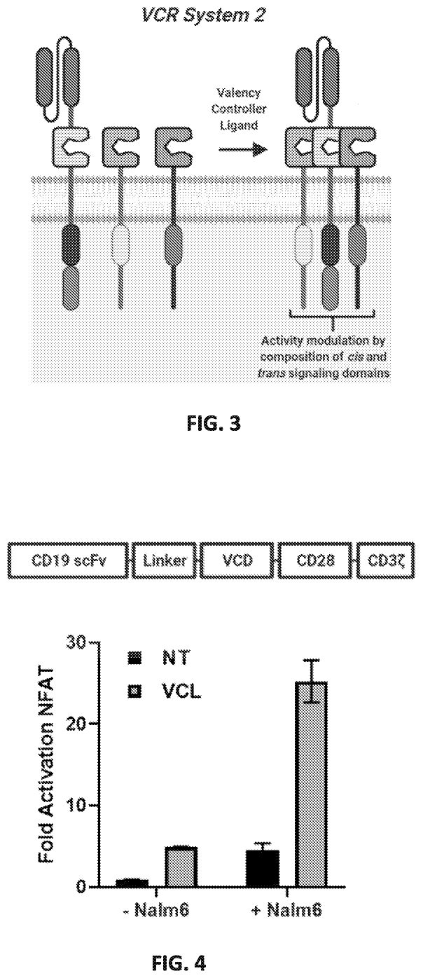

A valency controller module and valency controllable receptor CAR polypeptide were constructed as generally described in Example 1. The extramembrane signal recognition domain of the valency controllable receptor polypeptide was a CAR scFv that recognizes CD19. This extramembrane signal recognition domain was connected to a biotin-binding valency controller domain via a linker unit. In a T cell, the valency controller domain of the valency controllable receptor polypeptide spanned the cellular membrane. The valency controller domain was fused to a pair of CAR intramembrane signaling domains, CD28 and CD3ζ (CD247). The modified CAR was therefore of the type VCR-1928z and had a structure as schematically illustrated in the top of FIG. 4.

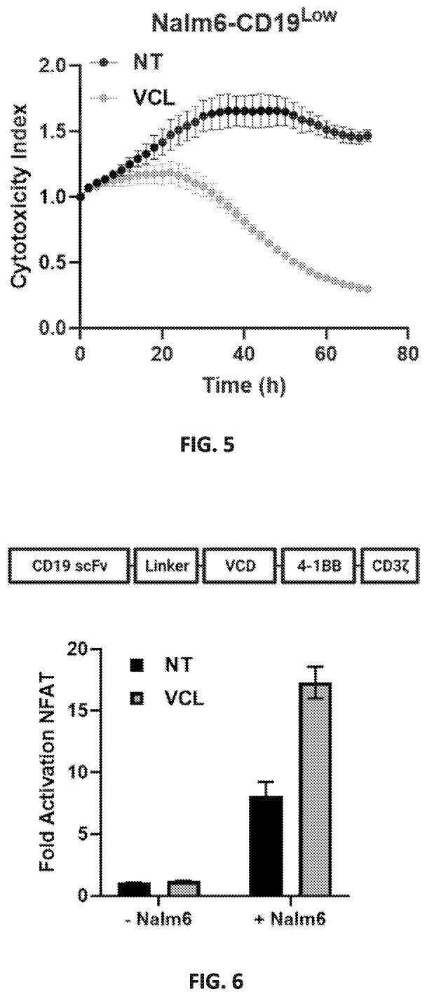

When the engineered receptor resides on the cell membrane, it induces oligomerization of the receptor upon binding the extracellular valency control ligand. The result of this oligomerization is enhanced signaling of intracellular nuclear factor of activated T-cells (NFAT)-dependent pathways that are central activation pathways of T cells. As shown in the graph of FIG. 4, this enhanced signaling in response to the valency control ligand was observed both in the absence (i.e., without scFv binding, priming) and in the presence (i.e., with scFv binding, synergizing) of CD19+ Nalm6 cells. Primary human T cells were engineered with the VCR-1928z receptor and shown to enhance T cell killing of target cells when treated with the valency control ligand (FIG. 5). These results demonstrate that the provided valency controllable receptor polypeptides and systems can improve the targeting of cells, e.g., the engineered Nalm6 cells of this example, that express only a relatively low density of a target antigen, e.g., CD19 in this example.

### Example 3. Construction of 2nd Generation CAR T Cells Modified with a Valency Controllable Receptor System

A valency controller module and valency controllable receptor CAR polypeptide were constructed as generally described in Examples 1 and 2. The extramembrane signal recognition domain of the valency controllable receptor polypeptide was a CAR scFv that recognizes CD19. This extramembrane signal recognition domain was connected to a biotin-binding valency controller domain via a linker unit. In a T cell, the valency controller domain of the valency controllable receptor polypeptide spanned the cellular membrane. The valency controller domain was fused to a pair of CAR intramembrane signaling domains, 4-1BB (CD137) and CD3ζ. The modified CAR was therefore of the type VCR-19BBz and had a structure as schematically illustrated in the top of FIG. 6.

When the engineered receptor resides on the cell membrane, it induces oligomerization of the receptor upon binding the extracellular valency control ligand. The result of this oligomerization is enhanced signaling of intracellular NFAT-dependent pathways that are central activation pathways of T cells. As shown in the graph of FIG. 6, this enhanced signaling in response to the valency control ligand was observed in the presence (i.e., with scFv binding, synergizing) of CD19+ Nalm6 cells. Primary human T cells were engineered with the VCR-19BBz receptor and shown to enhance T cell killing of target cells only when treated with the valency control ligand (FIG. 7). These results demonstrate that the provided valency controllable receptor polypeptides and ligands can in some instances enable strict external control of the targeting of cells, e.g., the engineered Nalm6 cells of this example, through the introduction of a valency control ligand.

### Example 4. Construction of a Valency Controllable Receptor System Leveraging DNA Origami to Control Ligand Valency

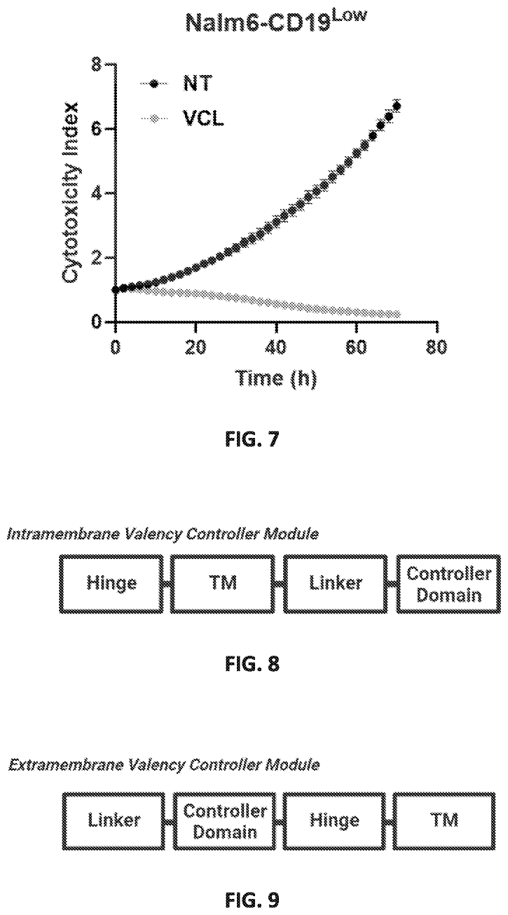

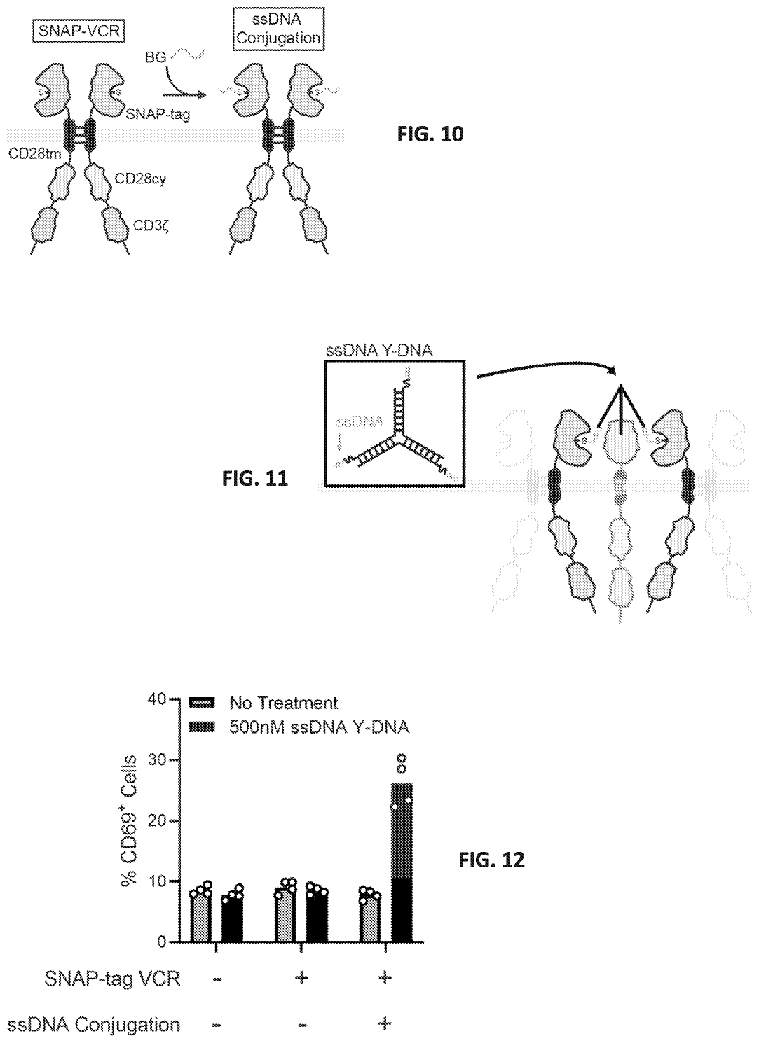

A nucleic acid-based DNA-receptor design was tested, wherein the design included a single stranded DNA (ssDNA) covalently conjugated to an extracellular SNAP-tag protein. The SNAP-tag was fused to the intracellular signaling domains (CD28 and CD3ζ) of a 2nd generation CAR connected by a IgG4 hinge and CD28 transmembrane domain (FIG. 10). This SNAP-VCR can interact with complementary ssDNA that is programmatically bound using Watson-Crick base pairing. DNA origami was leveraged to present the ssDNA by creating a rigid “Y” structure that is decorated at the tips of each of the three arms with ssDNA complementary to the DNA-conjugated SNAP-VCR (FIG. 11). Together, this system enables identification of clustering dynamics without the need for complex medicinal chemistry. Expressing the SNAP-VCR in a Jurkat cell line (JRTE6-1) demonstrates that clustering VCRs can induce low level T cell activation only when the binding domain (SNAP-tag conjugated with ssDNA) and ligand (DNA origami) are present (FIG. 12).

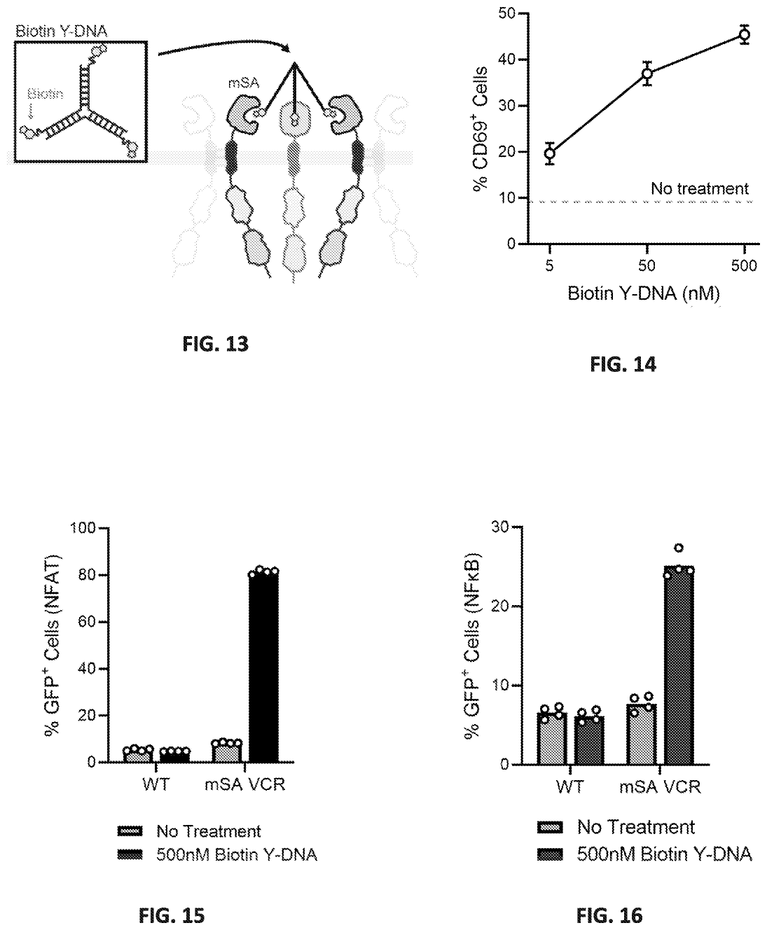

With the knowledge that DNA origami could be leveraged to control ligand valency, a more translatable system was developed by using the programmable valency of DNA origami as a method to present small molecule moieties. To do this the SNAP-tag domain was swapped for a monomeric streptavidin (mSA) domain on the VCR and the Y-DNA origami structure was decorated with biotin instead of ssDNA (FIG. 13). Jurkats expressing the mSA-VCR are tunably activated by treatment with biotin Y-DNA in a dose dependent manner (FIG. 14). Interestingly, clustering in the absence of mechanosensation was sufficient to induce both NFAT (FIG. 15) and nuclear factor kappa B (NFκB) (FIG. 16) signaling in Jurkat reporter lines.

### Example 5. Use of Programmable DNA Scaffolds to Prototype Drug Designs

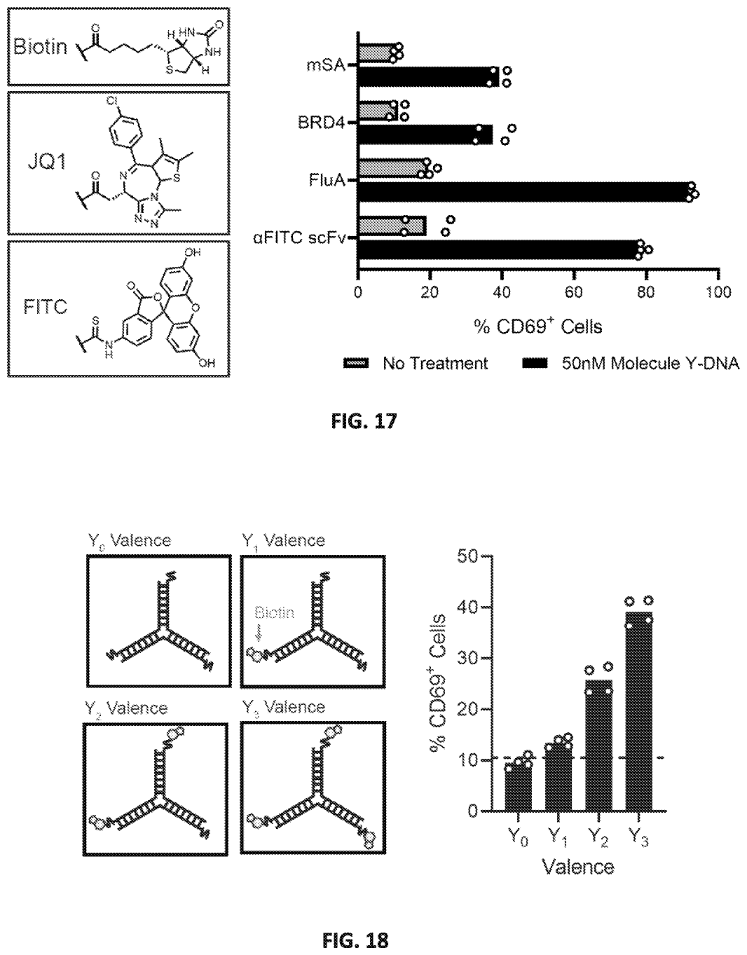

To further understand how drug design affects VCR-mediated T cell signaling, DNA origami scaffolds were used to determine which other small molecules/VCR pairs could be identified by conjugating candidate molecules to Y-DNA and fusing their binding domains to a VCR. The mSA-VCR described in Example 4 was domain swapped with a fluorescein isothiocyanate (FITC) binding single-chain variable fragment (scFv), FITC binding anticalin (FluA), or a human bromodomain-containing protein 4 (BRD4) domain, and Y-DNA origami was decorated with FITC or JQ1 (FIG. 17). All designs induced T cell activation only in the presence of the drug Y-DNA, however some drug-VCR pairs induced stronger activation than others. This demonstrates that receptor output could be tuned by simply altering the protein-ligand interactions.

To determine how receptor valency and therefore number of receptors in a cluster (cluster size) affected T cell activation the programmability of DNA origami was used to control the number of ligand moieties available to bind VCRs. A library of Y-DNA decorated with decreasing numbers of biotin molecules (valency=0, 1, 2, or 3) was synthesized. Induction of mSA-VCR expressing Jurkats by these valency scaffolds demonstrated that increased cluster size leads to increased T cell activation (FIG. 18).

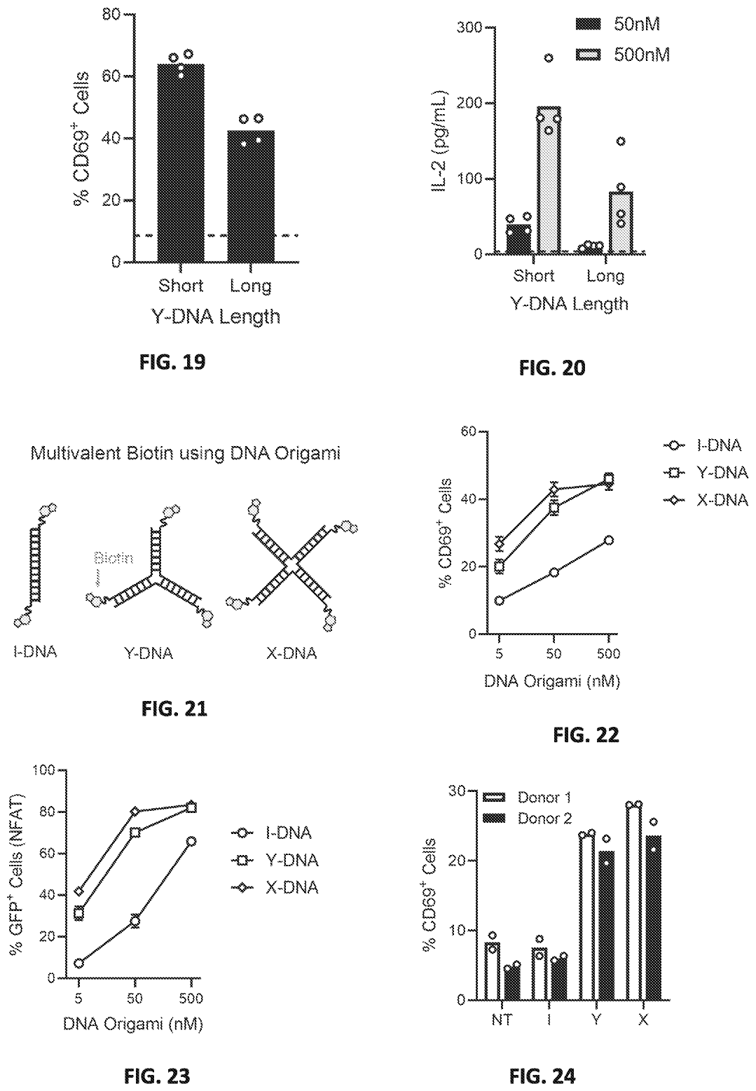

To test how proximity of drug-bound VCRs (cluster density) altered T cell activation, the length of the Y-DNA scaffold was altered by increasing the number of nucleotides in each of the arms from 20 to 36. Treatment with the shorter scaffold resulted in increased T activation (FIG. 19) indicating that increasing VCR proximity within clusters leads to more productive signaling. This proximity effect was further exaggerated when looking at activation-mediated IL-2 production in Jurkat cells (FIG. 20).

### Example 6. Translating DNA Scaffolds into Small Molecule Drugs

Ligand valency parameters were probed using three different DNA origami structures; I-DNA (2 biotins), Y-DNA (3 biotins), and X-DNA (4 biotins) (FIG. 21). Across three orders of ligand concentration, Y- and X-DNA generated comparable T cell activation and were both superior to I-DNA (FIG. 22). This activation pattern was also demonstrated in an NFAT reporter line (FIG. 23). We next asked if these design principles were also true in primary human T cells. In two different primary human T cell donors expressing an mSA-VCR, both Y- and X-DNA were comparable, and both performed substantially better than I-DNA (FIG. 24). This confirmed that in some circumstances it can be advantageous for an mSA-VCR ligand to have a valency greater than 2, and that VCR design rules that are developed in cell lines are translatable to primary human cells.

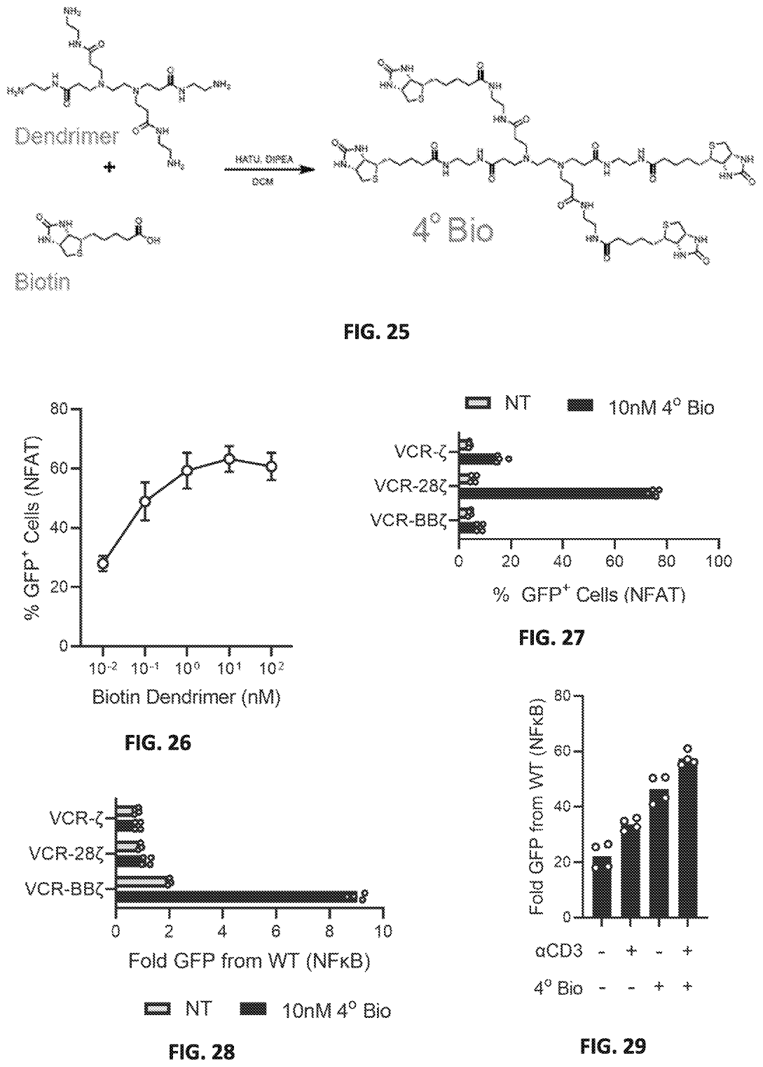

A dendrimer scaffold chemistry was then used to recapitulate the X- or Y-DNA design into a multivalent biotin drug-like molecule. While both the 3 and 4 valency designs performed similarly in the tested DNA system, the X design can be more accessible via commercially available scaffolds. Peptide coupling chemistry was used to covalently conjugate biotin to a generation 0 PAMAM dendrimer, converting the X-DNA origami hit into a lead small molecule (FIG. 25). This multivalent drug retains the ability for tunable clustering as demonstrated by activation of the NFAT pathway in Jurkat cells expressing mSA-VCR (FIG. 26).

Clinically relevant CAR designs contain other signaling domains in addition to the CD28 costimulatory domain. Further testing determined if the mSA-VCR system could activate T cells if it had a 4-1BB costimulatory domain. The VCR-28ζ induces strong NFAT signaling in the presence of drug (FIG. 27) while the VCR-BBζ induces strong NFκB signaling (FIG. 28). VCRs without a costimulatory domain also activate NFAT signaling but to a much lesser extent. To further extend the utility of the VCR platform. VCRs containing only the 4-1BB signaling domain were generated. Dendrimer-mediated clustering of this VCR-BB induced NFκB signaling and synergized with antibody-mediated CD3 signaling (FIG. 29). This indicates the potential for the VCR platform to control costimulatory signaling for applications beyond CAR T cells, for example, in combination with TCR T cell therapies.

### Example 7. Induction of VCR Signaling by Super-Clustering

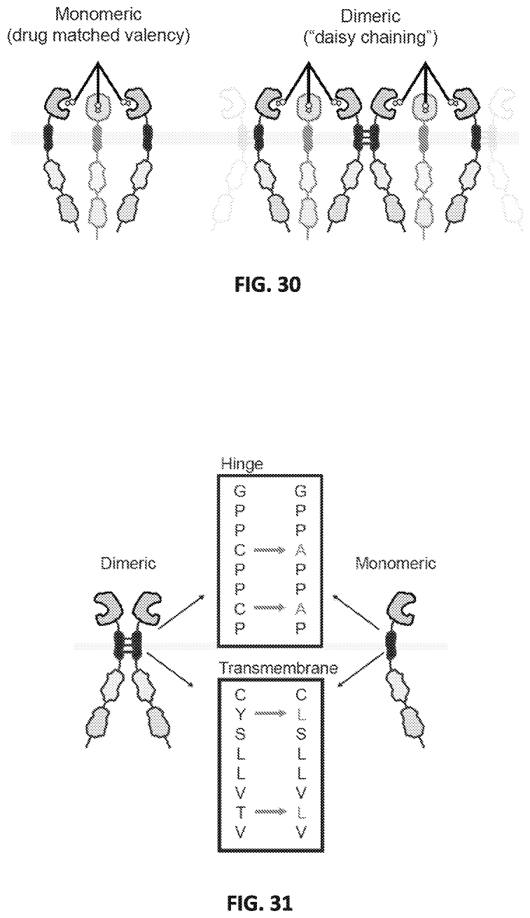

The IgG4 hinge and CD28 transmembrane domains used in the VCR system contain cysteine residues (S—S bonds) and hydrophobic residues (e.g., pi-pi stacking) leading to dimeric VCRs expressed on the surface of the cell. As a result, the drug may not simply create small clusters based on ligand valency (i.e., clusters of four VCRs in the tetravalent biotin case) but instead may induce “daisy-chaining” of many VCRs into large superclusters (FIG. 30). To demonstrate this mechanism, an mSA-VCR fused to a fluorescent protein (mSA-VCR-GFP) was expressed in HEK293T cells, and confocal microscopy was used to visualize the clusters with and without the tetravalent biotin (FIG. 31). In the presence of drug, the diffuse GFP membrane signal became large puncta residing on the cell surface indicating that the formation of large clusters was occurring.

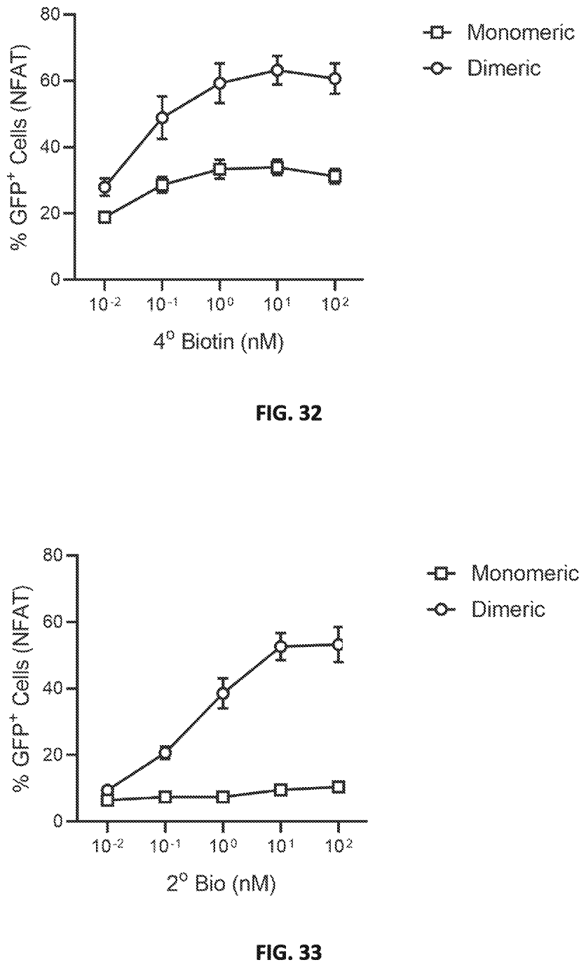

To further probe the effects of this super-clustering on signaling, the residues responsible for the cysteine bonds (C47A and C78A) in the hinge domain and hydrophobic interactions (Y89L and T109L) in the transmembrane domain were mutated into alanine and leucine residues, respectively, thereby engineering a monomeric version of mSA-VCR. A small molecule akin to the 1-DNA scaffold was generated to contain two biotins linked by a simple PEG linker. In the presence of the tetravalent biotin, the monomeric VCR had reduced capacity to signal in an NFAT reporter line (FIG. 32). In the presence of a bivalent biotin, the monomeric VCR loses all signaling capacity while the dimeric VCR is still able to respond (FIG. 33).

Based on these results the monomeric VCR's maximum cluster size is dictated by the ligand valency, while the dimeric VCR's cluster size is not limited by the ligand valency. Therefore, under some circumstances only dimeric VCRs treated with bivalent biotin are able to signal, indicating a daisy chaining mechanism must be occurring. Furthermore, the increased signaling of the dimeric VCR compared to the monomeric VCR when treated with the tetravalent biotin (valency=4) indicates that superclusters larger than four are being formed. Together with the microscopy data, these results indicate that in some embodiments the VCR design can form large super-clusters via daisy chaining, enabling enhanced signaling akin to TCR-based clustering.

### Example 8. Incorporating Antigen Binding into VCR

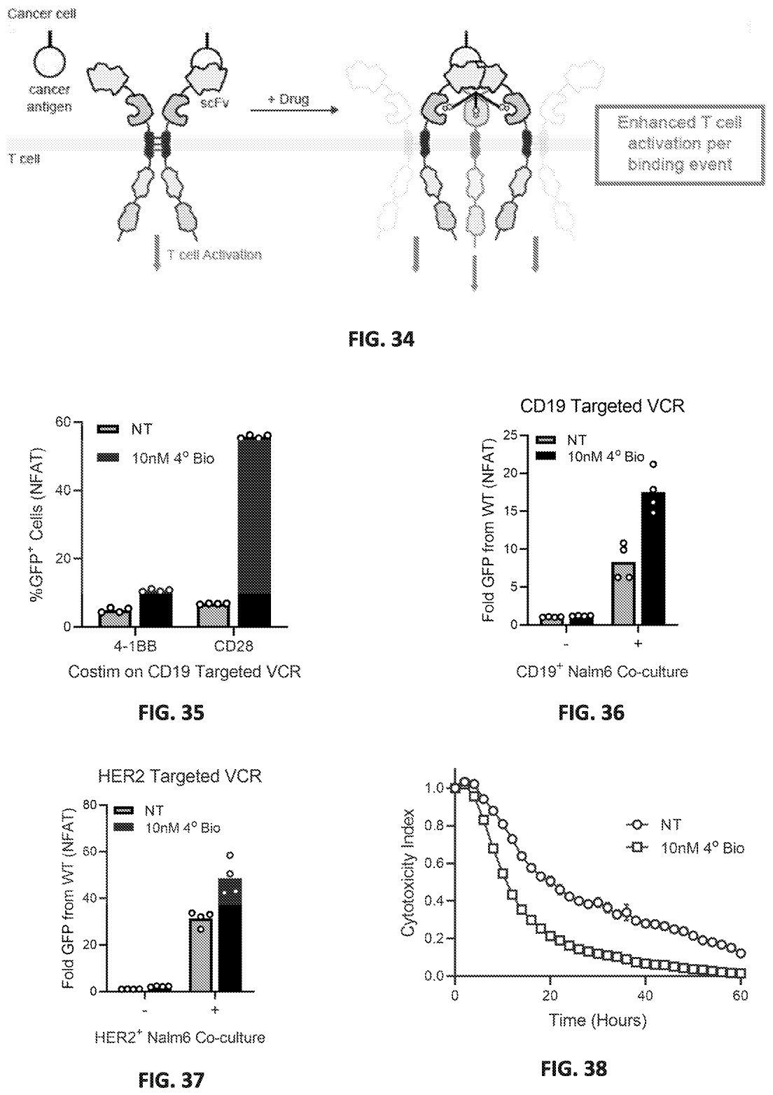

To fully recapitulate both the clustering and mechanosensitive aspects of a TCR, an scFv was incorporated into the extracellular portion of VCR (FIG. 34). In the absence of antigen, VCRs containing an mSA, a CD19 binding scFv, and the CD28 and CD3ζ signaling domains (VCR-1928z) displayed strong NFAT signaling in the presence of tetravalent biotin while the same VCR but with 4-1BB instead of CD28 (VCR-19BBz) has little to no NFAT signaling (FIG. 35). Recent data has shown that signaling through the NFAT pathway leads to exhaustion, thus VCR-19BBz was selected for further studies to limit the potential for drug-induced exhaustion in the absence of antigen.

NFAT reporter Jurkat cells engineered with VCR-19BBz were co-cultured with CD19+ Nalm6 cells with and without the tetravalent biotin (FIG. 36). Antigen targeted activity synergized with VCR clustering which was boosted nearly two-fold by treatment with the tetravalent biotin. This effect is not antigen dependent and can be observed when incorporating a HER2 scFv targeted toward HER2+ Nalm6 cell lines (FIG. 37).

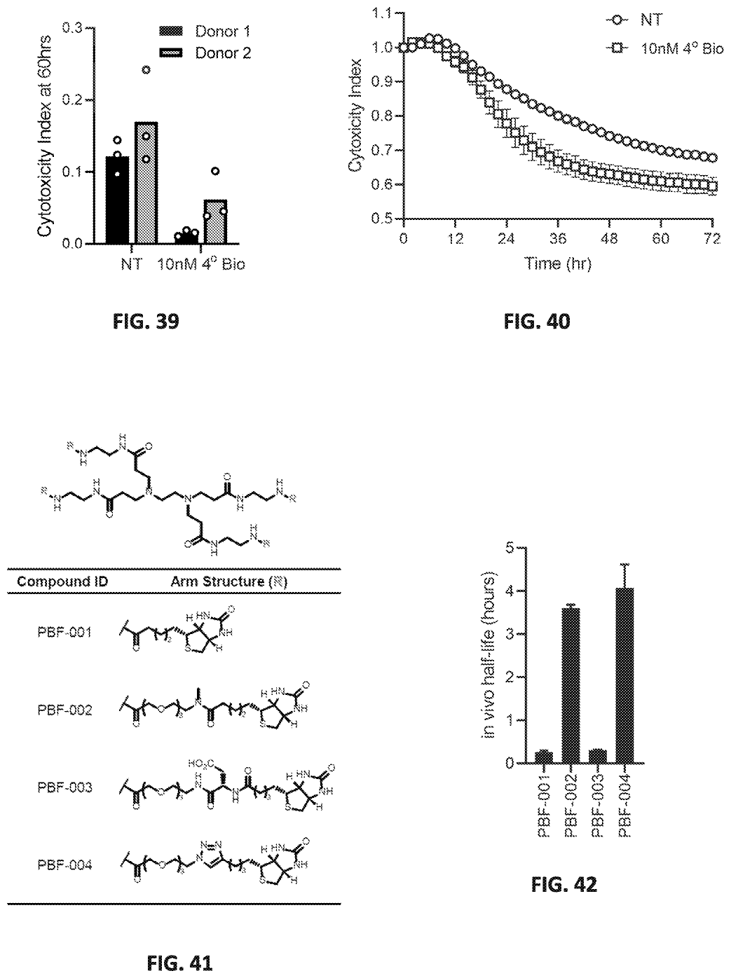

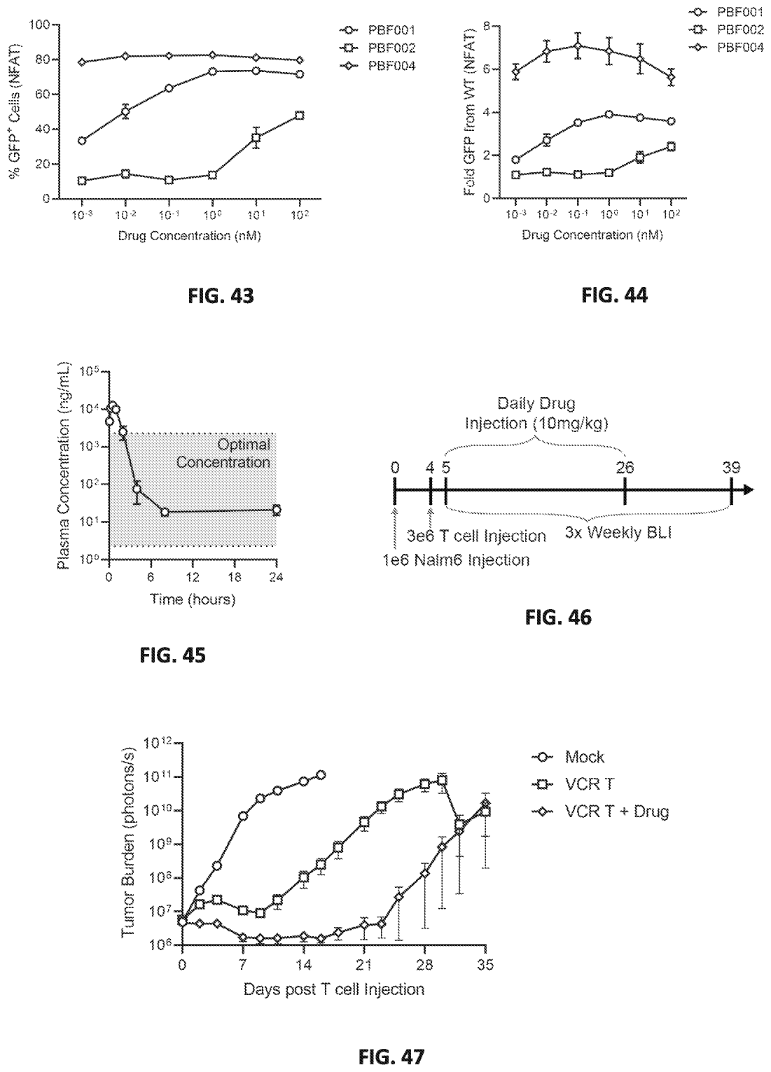

In primary T cells, drug-mediated boosting of antigen-dependent T cell activation enabled superior killing in vitro. Using live cell microscopy, fluorescently labeled CD19+ Nalm6 cells cultured with human primary VCR-19BBz T cells with and without tetravalent biotin were tracked, and the abundance of signal (integrated intensity) was quantified every two hours for 60 hours (FIG. 38). In two donors, inducing VCR clustering dramatically increased the killing ability of VCR T cells, as demonstrated by the final Nalm6 density measured at 60 hours (FIG. 39). Similarly, in a 143b spheroid model, VCR T cells targeting HER2 had increased killing activity over a 24-hour period (FIG. 41).

### Example 9. VCR Enhanced Cytotoxicity In Vivo Enabled by Lead Optimization

To identify a drug that would be sufficiently bioavailable in vivo, and thus useful as a therapy, a series of molecules were designed and synthesized (FIG. 41). This series was tested and the half-life of PBF002 and PBF004 was determined to be −4 hours, while the half-lives of PBF001 and PBF003 were less than 15 min (FIG. 42). Additional tests investigated whether the optimized leads could induce VCR clustering-mediated T cell activation in the NFAT Jurkat assay (FIG. 44). PBF004 performed better across three orders of magnitude compared to lead PBF001 while PBF002 performed worse and only at higher concentrations. Notably, PBF004 worked better at low concentrations. Plasma concentration analysis of PBF004 after intraperitoneal injection (i.p.) in wildtype mice over a 24-hour period indicated that drug concentration would be sufficient for VCR activation at a 10 mg/kg dose per day (FIG. 45). PBF004 can synergize with antigen targeting leading to a boost in VCR T cell cytotoxicity as measured by an in vitro killing assay.

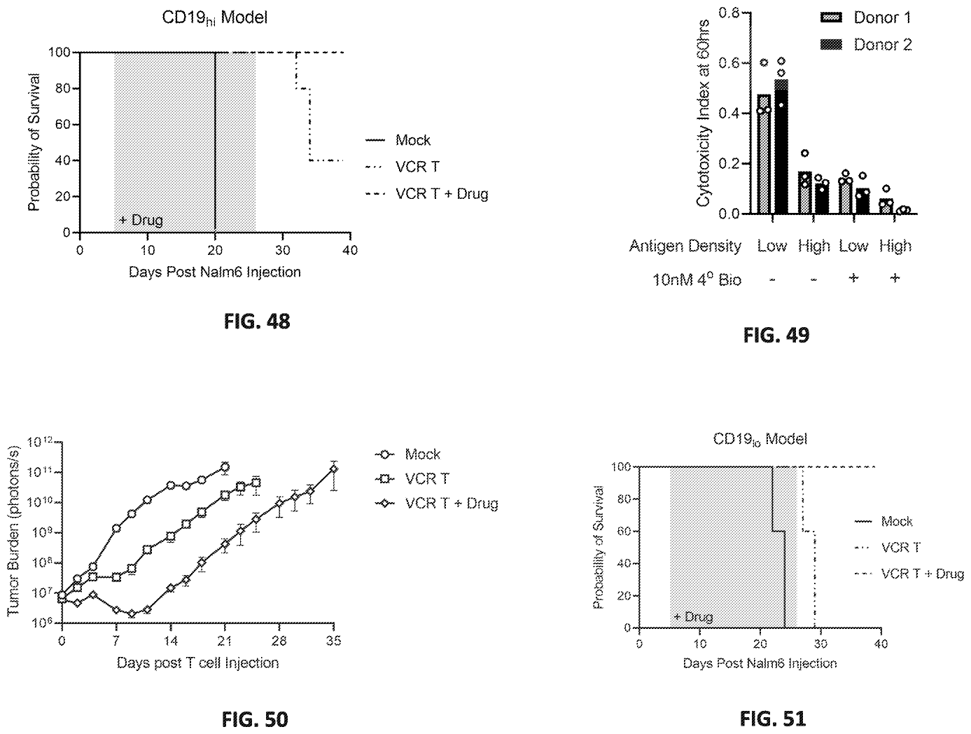

To test the VCR-CAR T cell activity with the VCR-19BBz receptor design and drug molecule in vivo, a mouse model of B cell cancer was generated by injecting 1×10⁶ luciferase-expressing CD19+ Nalm6 cells by tail vein injection into a NOD/SCID mouse (FIG. 46). Four days later, 3×10⁶ VCR-CAR+ T cells were injected intravenously into the tail vein. Mice were dosed with PBF004 at 10 mg/kg every day for a total of 21 days. Tumor burden was measured by BLI three times a week for 40 days. VCR-CAR T cell and drug-treated mice had significantly reduced tumor burden compared to both vehicle and uninduced VCR-CAR T cell mice (FIG. 47). In four of the five mice, tumors treated with VCR-CAR T cells and PBF004 had no apparent growth until drug dosing was removed at day 26. Importantly, drug treatment also conferred a survival benefit (FIG. 48).

### Example 10. Use of VCRs to Increase Sensitivity of T Cells to Antigen Low Cancers

The HER2+143b spheroid model is a low antigen density model. The enhanced cytotoxicity of drug activated VCR-CAR T cells toward this model indicated that clustering could sensitize T cells to low antigen density tumors. To see if the VCR platform could be utilized to overcome a common escape mechanism for CD19+ cancers targeted by CAR T cells, a recently developed CD19ₗₒ Nalm6 model was used. In an in vitro live cell microscopy assay, drug-induced VCR-CAR T cells vastly outperformed no induction after 60 hours of co-culture (FIG. 49).

To determine if this effect recapitulated in vivo, the in vivo model was repeated with the CD19ₗₒ Nalm6 line. Induced VCR-CAR T cells had reduced tumor burden and appeared to control the tumor for over a week, even though this model had been difficult to control in previous studies using clinically available CAR designs (FIG. 50). Additionally, all animals that had induced VCR-CAR T cells had a significant survival advantage while uninduced VCR-CAR T cells performed marginally better than mock but still all succumbed to the cancer.

Although the foregoing disclosure has been described in some detail by way of illustration and example for purpose of clarity of understanding, one of skill in the art will appreciate that certain changes and modifications within the spirit and scope of the disclosure may be practiced. e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the disclosure. In addition, each reference provided herein is incorporated by reference in its entirety for all purposes to the same extent as if each reference was individually incorporated by reference.

What is claimed is:

1. A valency controllable receptor polypeptide comprising:
an extramembrane signal recognition domain having an ability to recognize an extramembrane signal;
a valency controller module comprising:
a controller domain connected to the extramembrane signal recognition domain, wherein the controller domain is configured to recognize a custom input different from the extramembrane signal; and
a transmembrane domain connected to the controller domain;
wherein the valency controller module is configured to oligomerize the valency controllable receptor polypeptide upon recognition of the custom input by the controller domain; and
one or more intramembrane signaling domains connected to the transmembrane domain.

2. The valency controllable receptor polypeptide of claim 1, wherein the valency controller module further comprises:
one or more linker units connecting the one or more controller domains to the extramembrane domain.

3. The valency controllable receptor polypeptide of claim 1 or 2, wherein the valency controllable module further comprises:
one or more hinge domains connecting the one or more controller domains to the transmembrane domain.

4. The valency controllable receptor polypeptide of any one of claims 1-3, wherein the one or more intramembrane signaling domains each independently modulate one or more intramembrane pathways.

5. The valency controllable receptor polypeptide of claim 4, wherein at least one of the intramembrane signaling domains activates one or more intramembrane pathways.

6. The valency controllable receptor polypeptide of claim 4 or 5, wherein at least one of the intramembrane signaling domains inhibits one or more intramembrane pathways.

7. The valency controllable receptor polypeptide of any one of claims 4-6, wherein at least one of the intramembrane signaling domains enhances one or more intramembrane pathways.

8. The valency controllable receptor polypeptide of any one of claims 4-7, wherein at least one of the one or more intramembrane pathways is an endogenous pathway.

9. The valency controllable receptor polypeptide of any one of claims 4-8, wherein at least one of the one or more intramembrane pathways is an exogenous pathway.

10. The valency controllable receptor polypeptide of any one of claims 4-9, wherein at least one of the one or more intramembrane pathways is a synthetic pathway.

11. The valency controllable receptor polypeptide of any one of claims 4-10, wherein the one or more intramembrane pathways comprise genome sequence editing, transcription activation or repression, epigenetic modifications, genome translocation and rearrangement, RNA expression or degradation, RNA splicing or processing, post-transcription modifications of mRNA or ncRNA, post-translational modifications of proteins, cleavage or proteolysis of proteins, production or degradation of metabolites or other chemistries, antigen recognition or binding, trafficking of signaling molecules, cell cycle control, cell differentiation or reprogramming, T cell activation or exhaustion, programmed cell death, cell trafficking, secretion of cytokines or hormones, neuronal activity, macrophage phagocytosis, neutrophil NETpoptosis, immunological synapse formation, myeloid cell degranulation, antigen presentation, secretion or hypermutation of antibodies, production of oncolytic virus, or a combination thereof.

12. The valency controllable receptor polypeptide of any one of claims 1-11, wherein the custom input is an extramembrane input.

13. The valency controllable receptor polypeptide of any one of claims 1-11, wherein the custom input is an intramembrane input.

14. The valency controllable receptor polypeptide of any one of claims 1-13, wherein the custom input is a valency control ligand.

15. The valency controllable receptor polypeptide of claim 14, wherein the valency control ligand comprises a small molecule drug.

16. The valency controllable receptor polypeptide of claim 14 or 15, wherein the valency control ligand comprises a metabolite.

17. The valency controllable receptor polypeptide of any one of claims 14-16, wherein the valency control ligand comprises an oligonucleotide.

18. The valency controllable receptor polypeptide of any one of claims 14-17, wherein the valency control ligand comprises a peptide, a protein, a polysaccharide, a lipid, a glycoprotein, or a combination thereof.

19. The valency controllable receptor polypeptide of any one of claims 14-18, wherein the valency control ligand comprises an antibody, a nanobody, an scFv, a hormone, or a cytokine.

20. The valency controllable receptor polypeptide of any one of claims 14-19, wherein the valency control ligand comprises a polymer scaffold to which one or more interaction moieties are conjugated, wherein each of the one or more interaction moieties is independently a small molecule, a peptide, an oligonucleotide, a polysaccharide, or a lipid.

21. The valency controllable receptor polypeptide of any one of claims 1-13, wherein the custom input comprises a change in temperature or pH.

22. The valency controllable receptor polypeptide of any one of claims 1-13, wherein the custom input comprises a change in sound or electromagnetic radiation.

23. The valency controllable receptor polypeptide of any one of claims 1-13, wherein the custom input comprises a change in mechanical force.

24. The valency controllable receptor polypeptide of any one of claims 1-23, wherein the extramembrane signal recognition domain comprises a soluble N-ethylmaleimide-sensitive factor (NSF) attachment protein (SNAP) receptor domain, a biotin-binding domain, a leucine zipper domain, a single-chain variable fragment (scFv) domain, a nanobody domain, a cell-targeting domain from a natural polypeptide, or a variant or fragment thereof.

25. The valency controllable receptor polypeptide of any one of claims 1-24, wherein the controller domain comprises a small molecule binding domain.

26. The valency controllable receptor polypeptide of any one of claims 1-24, wherein the controller domain comprises an FK506 binding protein (FKBP) family domain, a bromodomain and extra terminal domain (BET) family domain, a gibberellin-insensitive dwarf (GID) family domain, a B-cell lymphoma 2 (Bcl-2) family domain, a soluble N-ethylmaleimide-sensitive factor (NSF) attachment protein (SNAP) receptor domain a biotin-binding domain, a nanobody, a single-chain variable fragment (scFv) domain, an E3 ubiquitin ligase domain, a natural or synthetic peptide epitope, or a variant or fragment thereof.

27. The valency controllable receptor polypeptide of any one of claims 1-26, wherein the one or more intramembrane signaling domains comprise a CD3ζ (CD247) domain.

28. The valency controllable receptor polypeptide of any one of claims 1-27, wherein the one or more intramembrane signaling domains comprise an immunoreceptor tyrosine-based activation motif (ITAM).

29. The valency controllable receptor polypeptide of claim 28, wherein the ITAM comprises a sequence from a CD37 domain, from a CD3S domain, from a CD3eε domain, from a DAP12 domain, from a CD32A domain, from a CD32C domain, from a CD132 domain, from a CD79a domain, from a CD79b domain, or from an FCER1G domain.

30. The valency controllable receptor polypeptide of any one of claims 1-29, wherein the one or more intramembrane signaling domains comprise a CD28 domain, a 4-1BB domain, an OX40 (CD134) domain, an ICOS (CD278) domain, a CD40 domain, a CD2 domain, or a CD27 domain.

31. A valency controllable receptor system comprising:
two or more valency controllable receptor polypeptides, each independently a valency controllable receptor polypeptide of any one of claims 1-30.

32. The valency controllable receptor system of claim 31, wherein the amino acid sequence of each of the two or more valency controllable receptor polypeptides is identical.

33. The valency controllable receptor system of claim 31, wherein the amino acid sequence of a first valency controllable receptor polypeptide of the two or more valency controllable receptor polypeptides differs from the amino acid sequence of a second valency controllable receptor polypeptide of the two or more valency controllable receptor polypeptides.

34. The valency controllable receptor system of claim 33, wherein at least one of the one or more intramembrane signaling domains of the first valency controllable receptor polypeptide differs from at least one of the one or more intramembrane signaling domains of the second valency controllable receptor polypeptide.

35. The valency controllable receptor system of claim 33 or 34, wherein the first valency controllable receptor polypeptide comprises a first controller domain configured to recognize a first custom input, and wherein the second valency controllable receptor polypeptide comprises a second controller domain configured to recognize a second custom input different from the first custom input.

36. The valency controllable receptor system of any one of claims 31-35, further comprising:
a membrane, wherein the transmembrane domain of each of the two or more valency controllable receptor polypeptides is located within the membrane.

37. The valency controllable receptor system of claim 36, wherein the membrane is a cellular membrane, a nuclear membrane, an organelle membrane, or a vesicle membrane.

38. The valency controllable receptor system of any one of claims 31-37, further comprising:
a valency control ligand, wherein the valency control ligand is the custom input recognized by the control domain of at least one of the one or more valency control polypeptides.

39. The valency controllable receptor system of any one of claims 31-38, further comprising:
an antigen, wherein the antigen is the extramembrane signal recognized by the extramembrane signal recognition domain of the one or more valency control polypeptides.

40. A valency controllable receptor system comprising:
a first valency controllable receptor polypeptides, wherein the first valency controllable receptor polypeptide is a valency controllable receptor polypeptide of any one of claims 1-30; and
a second valency controllable receptor polypeptide comprising:
a second valency controller module comprising:
a second controller domain configured to recognize a second custom input different from the extramembrane signal; and
a second transmembrane domain connected to the second controller domain:
wherein the second valency controller module is configured to oligomerize the second valency controllable receptor polypeptide upon recognition of the second custom input by the second controller domain; and
one or more intramembrane signaling domains connected to the second transmembrane domain.

41. The valency controllable receptor system of claim 40, wherein the second valency controllable receptor polypeptide does not comprise an extramembrane signal recognition domain having an ability to recognize an extramembrane signal different from the second custom input.

42. The valency controllable receptor system of claim 40 or 41, wherein the second valency controllable module is configured to oligomerize the first valency controllable receptor polypeptide with the second valency controllable receptor polypeptide upon recognition of the second custom input.

43. The valency controllable receptor system of any one of claims 40-42, wherein the second custom input is the same as the custom input recognized by the valency controller module of the first valency controllable receptor polypeptide.

44. The valency controllable receptor system of claim 43, wherein the amino acid sequence of the second valency controller module is the same as the amino acid sequence of the valency controller module of the first valency controllable receptor polypeptide.

45. The valency controllable receptor system of any one of claims 40-44, wherein at least one of the one or more intramembrane signaling domains of the second valency controllable receptor polypeptide differs from at least one of the one or more intramembrane signaling domains of first valency controllable receptor polypeptide.

46. The valency controllable receptor system of any one of claims 40-45, further comprising:
a membrane, wherein the second transmembrane domain and the transmembrane domain of the first valency controllable receptor polypeptides are located within the membrane.

47. The valency controllable receptor system of claim 46, wherein the membrane is a cellular membrane, a nuclear membrane, an organelle membrane, or a vesicle membrane.

48. The valency controllable receptor system of any one of claims 40-47, further comprising:
a valency control ligand, wherein the valency control ligand is the custom input recognized by the second valency control domain or the valency control domain of the first valency controllable receptor polypeptide.

49. The valency controllable receptor system of any one of claims 40-48, further comprising:
an antigen, wherein the antigen is the extramembrane signal recognized by the extramembrane signal recognition domain of the first valency controllable receptor polypeptide.

50. A host cell comprising the valency controllable receptor polypeptide of any one of claims 1-30, or the valency controllable receptor system of any one of claims 31-49.

51. The host cell of claim 50, wherein the host cell is a lymphocyte, a phagocytic cell, a granulocytic cell, or a dendritic cell.

52. The host cell of claim 51, wherein the lymphocyte is a T cell, a B cell, a natural killer (NK) cell, or an innate lymphoid cell (ILC).

53. The host cell of claim 52, wherein the T cell is a CD4⁺ helper αβT cell, a CD8⁺ killer αβT cell, a δγT cell, or a natural killer T (NKT) cell.

54. The host cell of claim 51, wherein the phagocytic cell is a monocyte or a macrophage.

55. The host cell of claim 51, wherein the granulocytic cell is a neutrophil, a basophil, an eosinophil, or a mast cell.

56. The host cell of claim 50, wherein the host cell is a stem cell or a progenitor cell.

57. The host cell of claim 56, wherein the stem cell is an induced pluripotent stem cell (iPSC), an embryonic stem cell (ESC), an adult stem cell, or a mesenchymal stem cell (MSC).

58. The host cell of claim 56, wherein the progenitor cell is a neural progenitor cell, a skeletal progenitor cell, a muscle progenitor cell, a fat progenitor cell, a heart progenitor cell, a chondrocyte, a fibroblast cell, or a pancreatic progenitor cell.

59. The host cell of claim 50, wherein the host cell is a progenitor-cell-differentiated cell, a stem-cell-differentiated cell, an organoid, or an assembloid.

60. A method for modulating an intramembrane pathway, the method comprising providing the valency controllable receptor polypeptide of any one of claims 1-30, or the valency controllable receptor system of any one of claims 31-49.

61. The method of claim 60, further comprising exposing the valency controllable receptor polypeptide to the custom input.

62. A method for preventing or treating a disease in a subject, the method comprising administering to the subject an amount of the valency controllable receptor polypeptide of any one of claims 1-30, the valency controllable receptor system of any one of claims 31-49 or the host cell of any one of claims 50-59.

63. The method of claim 62, further comprising, subsequent to the administering, exposing the valency controllable receptor polypeptide to the custom input.

64. The method of claim 63, wherein the exposing comprises introducing to the subject a therapeutically effective amount of the custom input.

65. The method of any one of claims 62-64, wherein the disease is a cancer.

66. The method of any one of claims 62-64, wherein the disease is a cancerous tumor.

67. The method of claim 66, wherein the cancerous tumor is a solid cancerous tumor.

68. The method of claim 66, wherein the cancerous tumor is a liquid cancerous tumor.

69. The method of any one of claims 62-64, wherein the disease is an infectious disease.

70. The method of claim 69, wherein the infectious disease is a viral infectious disease or a bacterial infectious disease.

71. The method of any one of claims 62-64, wherein the disease is an autoimmune disease.

72. The method of any one of claims 62-64, wherein the disease is an age-related disease.

73. The method of any one of claims 62-64, wherein the disease is a neurological disease.

74. A method for healing a wound in a subject, the method comprising administering to the subject an amount of the valency controllable receptor polypeptide of any one of claims 1-30, or the valency controllable receptor system of any one of claims 31-49, or the host cell of any one of claims 50-59.

75. The method of claim 74, further comprising, subsequent to the administering, exposing the valency controllable receptor polypeptide to the custom input.

76. The method of claim 75, wherein the exposing comprises introducing to the subject a therapeutically effective amount of the custom input.
